(19) United States
(12) Patent Application Publication
(10) Pub. No.: US 2009/0156968 A1
(43) Pub. Date: Jun. 18, 2009

(54) METHOD FOR EVALUATING AND TREATING MUSCULOSKELETAL DYSFUNCTIONS

(76) Inventor: Aleyna Devi Sheradha, Lethbridge (CA)

Correspondence Address: TOWNSEND AND TOWNSEND AND CREW, LLP, TWO EMBARCADERO CENTER, EIGHTH FLOOR, SAN FRANCISCO, CA, 94111-3834, US

(21) Appl. No.: 12/337,428

(22) Filed: Dec. 17, 2008

(30) Foreign Application Priority Data
Dec. 18, 2007 (CA) 2,615,179

(51) Int. Cl. A61H 1/00; A61B 19/00

(52) U.S. Cl. 601/1; 128/898

(57) ABSTRACT

There are disclosed methods and apparatus for evaluating and for treating a musculoskeletal dysfunction in a human. The methods may comprise carrying out functional assessments or may comprise repositioning body tissues.

### FIELD

The embodiments disclosed relate to methods of evaluating musculoskeletal dysfunctions, treating musculoskeletal dysfunctions, and improving musculoskeletal performance.

### BACKGROUND

The prior art discloses a range of methods for manipulating musculoskeletal tissues. U.S. Pat. No. 6,090,045 to Leahy et al., U.S. Pat. No. 6,283,916 Leahy et al., and U.S. Pat. No. 6,491,651 Leahy et al. disclose methods for reducing the size and effect of various adhesions or lesions in soft tissues.

### SUMMARY

In a first embodiment there is disclosed a method for treating a musculoskeletal dysfunction in a body region of a human, the body region comprising a first tissue and having a first configuration, the method comprising the steps of: a) carrying out a first functional assessment comprising: i) causing the human to actuate the body region; ii) observing predetermined features of the actuation of the body region; and iii) comparing the predetermined features of the observed actuation to the predetermined features of a desired actuation to assess the musculoskeletal dysfunction; and b) repositioning the first tissue in a second configuration to treat the musculoskeletal dysfunction.

In alternative embodiments the actuation comprises holding the body region substantially static and the functional assessment further comprises observing the tissue actuation required to maintain the stasis.

In alternative embodiments the predetermined feature may be a loading pattern of the body region.

In alternative embodiments the body region comprises a second tissue and the repositioning comprises repositioning of the first tissue relative to the second tissue to establish a substantially stable second configuration.

In alternative embodiments the method may further comprise a) conducting a second functional assessment; and b) comparing the predetermined features of the actuation during the first functional assessment with the predetermined features of the actuation during the second functional assessment.

In alternative embodiments the first tissue has a length and the repositioning comprises applying pressure substantially perpendicular to the length.

In alternative embodiments the method further comprises training the body region to adopt the second configuration.

In alternative embodiments the training comprises guiding a movement of the body region to follow a predetermined pattern while causing the human to concentrate on the quality of the guided movement.

In alternative embodiments the treatment has a long term efficacy.

In alternative embodiments the functional assessment comprises the using an apparatus to monitor the predetermined features of the actuation.

A method for evaluating a musculoskeletal dysfunction in a body region of a human, the body region having a tissue configuration, the method comprising: a) causing the human to actuate the body region; b) observing predetermined features of the actuation of the body region; and c) comparing the predetermined features of the observed actuation to predetermined features of a desired actuation to evaluate the musculoskeletal dysfunction.

In alternative embodiments the method further comprises training the body region substantially static and the functional assessment further comprises observing the tissue actuation required to maintain the stasis.

In alternative embodiments the predetermined feature is a loading pattern of the body region.

In alternative embodiments the body region comprises a second tissue and the repositioning comprises repositioning of the first tissue relative to the second tissue to establish substantially stable second configuration.

In alternative embodiments the method may further comprise a) conducting a second functional assessment; and b) comparing the predetermined features of the actuation during the first functional assessment with the predetermined features of the actuation during the second functional assessment.

In alternative embodiments the first tissue has a length and the repositioning comprises applying pressure substantially perpendicular to the length.

In alternative embodiments the method may further comprise training the body region to adopt the second configuration.

In alternative embodiments the training comprises guiding a movement of the body region to follow a predetermined pattern while causing the human to concentrate on the quality of the guided movement.

In alternative embodiments the treatment has a long term efficacy.

In alternative embodiments the functional assessment comprises using an apparatus to monitor the predetermined feature of the actuation.

A method for enhancing the performance of a body region of a human, the body region having a first configuration and a first tissue, the method comprising evaluating the actuation of the body region prior to repositioning the first tissue, the evaluating comprising: a) causing the human to actuate the body region; b) observing predetermined features of the actuation of the body region; c) comparing the predetermined features of the observed actuation to predetermined features of a desired actuation to assess the musculoskeletal dysfunction.

In alternative embodiments the actuation comprises holding the body region substantially static and the functional assessment further comprises observing the tissue actuation required to maintain the stasis.

In alternative embodiments the predetermined feature is a loading pattern of the body region.

In alternative embodiments the body region comprises a second tissue and the repositioning comprises repositioning of the first tissue relative to the second tissue to establish a substantially stable second configuration.

In alternative embodiments the method may further comprise a) conducting a second functional assessment; and b) comparing the predetermined features of the actuation during the first functional assessment with the predetermined features of the actuation during the second functional assessment.

In alternative embodiments the first tissue has a length and the repositioning comprises applying pressure substantially perpendicular to the length.

In alternative embodiments the method may further comprise training the body region to adopt the second configuration.

In alternative embodiments the training comprises guiding a movement of the body region to follow a predetermined pattern while causing the human to concentrate on the quality of the guided movement.

In alternative embodiments the treatment has a long term efficacy.

In alternative embodiments the functional assessment comprises the use of an apparatus to monitor the predetermined features of the actuation.

A method for training others in use of a guide to treat a musculoskeletal dysfunction in a body region of a human, the body region comprising a first tissue and having a first configuration, the method comprising the steps of: a) training others to use the guide to carry out a first functional assessment comprising: i) causing the human to actuate the body region; ii) observing predetermined features of the actuation of the body region; and iii) comparing the predetermined features of the observed actuation to the predetermined features of a desired actuation to assess the musculoskeletal dysfunction; and b) training others to use the guide to reposition the first tissue in a second configuration to treat the musculoskeletal dysfunction.

In alternative embodiments the actuation comprises holding the body region substantially static and the functional assessment further comprises observing the tissue actuation required to maintain the stasis.

In alternative embodiments the predetermined feature is a loading pattern of the body region.

In alternative embodiments the body region comprises a second tissue and the repositioning comprises repositioning of the first tissue relative to the second tissue to establish a substantially stable second configuration.

In alternative embodiments the method may further comprise a) conducting a second functional assessment; and b) comparing the predetermined features of the actuation during the first functional assessment with the predetermined features of the actuation during the second functional assessment.

In alternative embodiments the first tissue has a length and the repositioning comprises applying pressure substantially perpendicular to the length.

In alternative embodiments the method may further comprise training the body region to adopt the second configuration.

In alternative embodiments the training comprises guiding a movement of the body region to follow a predetermined pattern while causing the human to concentrate on the quality of the guided movement.

In alternative embodiments the treatment has a long term efficacy.

In alternative embodiments the functional assessment comprises using an apparatus to monitor the predetermined features of the actuation.

An apparatus for determining an actuation pattern of predetermined musculoskeletal tissues in a human subject having the musculoskeletal tissues.

In alternative embodiments the method may further comprise the use of the apparatus to determine the effectiveness of the repositioning in treating a musculoskeletal dysfunction.

In alternative embodiments the method may further comprise use of the apparatus according to other embodiments to assess musculoskeletal performance.

Features and advantages of the subject matter disclosed will become more apparent in light of the following detailed description of some embodiments thereof, as illustrated in the accompanying figures. As will be realized, the various embodiments are capable of modifications in various respects and may be combined in a variety of alternative ways, all without departing from the spirit and scope of the claims. Accordingly, the drawings and the description are to be regarded as illustrative in nature, and not as restrictive.

### BRIEF DESCRIPTION OF THE DRAWINGS

FIG. 1 is an assessment according to the first embodiment;

FIG. 2 is a further assessment according to the first embodiment;

FIG. 3 is an intervention according to the first embodiment;

FIG. 4 is a second illustration of an intervention according to the first embodiment;

FIG. 5 is a third illustration of an intervention according to the first embodiment;

FIG. 6 is an assessment according to Example 1;

FIG. 7 is an assessment according to Example 1;

FIG. 8 is an illustration of an intervention according to Example 1;

FIG. 9 is a second illustration of an intervention according to Example 1;

FIG. 10 is a third illustration of an intervention according to Example 1;

FIG. 11 is a fourth illustration of an intervention according to Example 1,

FIG. 12 is an assessment according to Example 2;

FIG. 13 is; a further assessment according to Example 2;

FIG. 14 is a first illustration of an intervention according to Example 2;

FIG. 15 is a second illustration of an intervention according to Example 2;

FIG. 16 is a third illustration of an intervention according to Example 2;

FIGS. 17A and B is show an intervention according to Example 2;

FIG. 18 is a sixth illustration of an intervention according to Example 2;

FIG. 19 is an assessment according to Example 3;

FIG. 20 is a further assessment according to Example 3;

FIG. 21 is a first illustration of an intervention according to Example 3;

FIG. 22 is a second illustration of an intervention according to Example 3;

FIG. 23 is an intervention according to Example 3;

FIG. 24 is an intervention according to Example 4;

FIG. 25 is a further illustration of an intervention according to Example 4;

FIG. 26 is an assessment according to Example 5;

FIG. 27 is a first illustration of an intervention according to Example 5;

FIG. 28 is a second illustration of an intervention according to Example 5;

FIG. 29 is a third illustration of an intervention according to Example 5;

FIG. 30 is a fourth illustration of an intervention according to Example 5;

FIGS. 31A and B are assessments according to Example 6;

FIG. 32 is a first illustration of intervention according to Example 6;

FIG. 33 is a second illustration of intervention according to Example 6;

FIG. 34 is a third illustration of intervention according to Example 6;

FIG. 35 is a fourth illustration of intervention according to Example 6;

FIGS. 36 A and B show assessments according to Example 7;

FIG. 37 is an illustration of an intervention according to Example 7;

FIG. 38 shows the postures for assessments according to Example 8;

FIG. 39 is a first illustration of an intervention according to Example 8;

FIG. 40 is a second illustration of an intervention according to Example 8;

FIG. 41 is a third illustration of an intervention according to Example 8;

FIG. 42 is an assessment according to Example 9;

FIG. 43 is an illustration of an intervention according to Example 9;

FIG. 44 shows assessments according to Example 10;

FIG. 45 is a first illustration of an intervention according to Example 10;

FIG. 46 is a second illustration of an intervention according to Example 10;

FIG. 47 is a third illustration of an intervention of Example 10;

FIG. 48 is a fourth illustration of an intervention according to Example 10;

FIG. 49 is a fifth illustration of an intervention according to Example 10;

FIG. 50 is an illustration of the use of an apparatus of an embodiment.

### DETAILED DESCRIPTION OF EMBODIMENTS

### Definitions

In this disclosure the following terms have the following meanings which are presented by way of illustration and clarification only and are not limiting:

In this disclosure the term “active movement” is intended to mean musculoskeletal movement actuated by the subject to be treated, evaluated, or otherwise assisted using the methods set out herein.

In this disclosure the term “alignment” is intended to mean the relative arrangement or position of tissues or parts of a body region or the act of adjusting the relative arrangement or position of tissues, as the context requires.

In this disclosure the term “body region” is intended to mean any part of a body and may include appendages such as arms, legs, hands or feet, digits, or neck, and may include other body regions such as regions comprising one or more of back, upper back, mid back, lower back, abdomen, thorax, trunk, rib cage, anterior or posterior pelvis, anterior or posterior leg, upper or lower leg, ankle, foot, tarsal region, upper arm or forearm, wrist, hand, carpal region, mid back, the core axis of the body, anterior or posterior shoulder, upper trunk, mid trunk, lower trunk, upper and lower abdomen, anterior and posterior abdomen, soft tissue groupings, muscle groupings, connective tissue groupings, skeletal groupings, shoulder girdle, pelvic girdle, joint regions. A body region may comprise any one or more of the foregoing or sub parts of any of the foregoing in any suitable combination as will be readily apparent and understood by those skilled in the art.

In this disclosure the term “configuration” is intended to have its ordinary meaning but for greater certainty it is intended to include the relative arrangement or position of tissues or parts of a body region and includes terms such as alignment, conformation, arrangement, cooperation, and includes both configuration at rest and while in motion.

In this disclosure the term “desired actuation” is intended to mean to any form or aspect of the actuation of musculoskeletal tissues or any portion of a body region which is determined to reflect selected desired characteristics including but not limited to patterns of nervous activity, muscle movement, tension or contraction, posture, muscle loading, muscle positioning; muscle innervation, muscle alignment, tissue configuration and the like. In alternative embodiments such desired characteristics and the determination of any degree of conformity of any portion of the musculoskeletal system thereto may be determined directly by observation by a practitioner or may be determined using a suitable apparatus, manual or guide, database, expert system or other suitable aid.

In this disclosure the term “evaluate” is intended to have its normal meaning and to include activities such as assessing, diagnosing, determining, analysing, measuring, visualising, reducing to electronic data, modelling, observing, proposing explanations for and/or responses to or otherwise characterising a set of circumstances, properties or data.

In this disclosure the term “guide” where used as a noun is intended to mean any device, object, system or resource that offers basic information or instructions to supervise the training for others to carry out methods or parts of methods or aspects of methods such as those disclosed herein. Examples of a guide may include but are in no way limited to a book, a manual, a pamphlet; a DVD, CD, tape, digital information, computer software, an electronic expert system, a device handheld or otherwise, a programmed computer, a web based or web accessible system, or any other device, system or collection of data and/or instruction useable to assist a practitioner or user to implement any aspect of the subject matter disclosed herein.

In this disclosure the term “length” is intended to have its normal meaning and to include the distance between any suitable reference points, such as the distance between insertion points at the ends of a particular muscle, or such other distances as may be suitable for the evaluation or assessment in question. A range of suitable lengths for particular purposes will readily identified by those skilled in the art with the guidance provided in this disclosure.

In this disclosure the term “long term efficacy” is intended to mean conferring long-term relief from the musculoskeletal dysfunction and in particular alternative embodiments may mean that once any course of treatment is completed or is substantially completed, the subject may be relieved, or substantially relieved, of the dysfunction for a period of up to about three months, six months, a year, two years, three years, four years, five years, six years, seven years, eight years or longer with only relatively few instances of repeated treatment being necessary to maintain such relief.

In this disclosure the term “mammal” is intended to have its ordinary meaning and includes humans.

In this disclosure the term “method” is intended to include any method, system, way, strategy, process, procedure, technique, manner or mode of doing something or carrying out any activity, and in alternative embodiments any method may be implemented directly by a practitioner or with the assistance of any artificial or man made devices or systems including computers and software.

In this disclosure the term “monitor” is intended to include any form of monitoring, observing, detecting, recording, measuring, observing or evaluating, assessing, diagnosing or, as the context requires, it may also refer to any form of device or apparatus for displaying data or information for a user.

In this disclosure the term “musculoskeletal” is intended to include combinations of one or more of any or all types of body tissues, and includes but is not limited to muscles, bones, connective tissue, tendons, ligaments, synovial tissue, capsules, fascia, smooth and striated muscle, skin and fat. In particular embodiments musculoskeletal tissue may include other tissue types such as blood vessels, fat, areolar tissue, nervous tissue and other tissue types all of which will be readily understood by those skilled in the art.

In this disclosure the term “musculoskeletal dysfunction” is intended to mean all forms of sub-optimal or abnormal musculoskeletal function or structure. It may comprise any kind of chronic or acute tissue damage, trauma, disease, or disorder and may include neuropathies and may result from any cause including acute injury, trauma, disease, or any genetic or other condition. In particular embodiments it may include tendonitis, tenosynovitis, bursitis, impingement on spinal nerves which may for example result from a protruding or prolapsed intervertebral disc, or may include carpal tunnel syndrome, plantar fasciitis, rotator cuff disorders, knee compartment syndrome, SI joint pain, hip joint pain, neck and shoulder pain, thoracic pain, repetitive stress injuries. It may include situations where a function or a structure is normal relative to a sample of the general population but where some improvement in performance is or may be achievable by the methods disclosed and claimed herein.

In this disclosure the term “notice” or “to notice” is intended to mean the focussing of particular attention on or paying particular attention to a circumstance and instructions to a subject to “notice” a particular pattern of actuation or tissue configuration may for example include drawing the subjects attention to sensations associated with one or more features of muscle actuation, tension, position, comfort or other aspects of body function or may include asking the subject questions about such sensations.

In this disclosure the term “observe” is intended to mean all types of observation, assessment, evaluation, recording, monitoring, diagnosing, analysis, measurement and the like and in particular embodiments it may include the use of artificial apparatuses or devices of any kind to monitor, record, model, assess, evaluate, record, or otherwise characterise data. It includes both direct visual observation, and observations made through physical manipulation or other sensing and in embodiments it may relate to aspects of the position, configuration, actuation or other properties of a body, body region or tissue. Observing may comprise or consist of directly or indirectly, with or without the aid of mechanical, electronic or other apparatuses or devices, visually or otherwise, measuring or making any qualitative or quantitative evaluation, assessment or other characterisation or accumulating a data set.

In this disclosure the term “passive movement” is intended to mean musculoskeletal movement of a subject wherein the subject does not itself actuate the movement but allows a practitioner to move the relevant body part of the subject.

In this disclosure the term “predetermined feature” means any aspect or feature of the actuation of the body region in question as well as related regions of the body, and may include such features as muscle tone, stability, tremor, patterns of muscle activation, rate of fatigue, intensity or localisation of pain, discomfort or other muscle loading sensations such as the recruitment of compensation muscles or muscle groups, overall strength, recruitment of inappropriate or sub optimal muscles or tissues, posture, orientation of one or more tissues, nerve activation, and may extend to the analysis of such features outside of the specific body region under consideration. Suitable features of any body region for particular embodiments, and the diagnostic properties of such features, will be readily understood by those skilled in the art in light of the guidance presented in this disclosure. In alternative embodiments the term “predetermined feature” may include one, two, three, four or more aspects or features and may relate to different body regions or to combinations of more than one body region.

In this disclosure the term “tissue” is intended to mean any type of body tissue or any combination of types of body tissue that may be encompassed within the term “musculoskeletal”, where the terms “first tissue” and “second tissue” are used, it will be understood that these may be the same or different as the context requires.

In this disclosure the term “treatment” (or “treating”) is intended to include its normal meaning and to include without limitation such activities as improving, alleviating, controlling, preventing, minimising, manipulating, adjusting, or aligning or re-aligning, positioning, re-positioning, configuring, or re-configuring tissues, with the goal or result of preventing, ameliorating, reducing, or correcting a musculoskeletal dysfunction, and includes any activity that may cause, permit, promote, facilitate or assist any improvement in musculoskeletal function.

The embodiments of the invention are described with reference to the accompanying FIGS. 1 through 50 which are illustrative of particular embodiments and are not limiting. For clarity, not all structures are numbered in all drawings, and alternative illustrations may be numbered to draw attention to different features of an embodiment. Some illustrations may have been simplified for clarity of explanation.

### First Embodiment

In a first embodiment generally illustrated with reference to FIGS. 1 through 5 there is disclosed a method for treating a musculoskeletal dysfunction in a body region of a mammal which may be a human. The body region may be or may comprise a leg or part of a leg 100 and may comprise the muscles of the anterior leg 110 together with any associated muscles that may be recruited from time to time. The body region may comprise a first tissue and have a first configuration. The first tissue may comprise one or more of a number of muscles or muscle groups and the muscles may comprise one or more muscles comprising the rectus femoris, the sartorius, the tensor fascia lata, and the three aspects of the vastas in the thigh, and the tibialis anterior and the extensor hallucis longus in the shank or may comprise other muscles that may compensate for deficiencies in this group. In FIGS. 1 through 5 the quad/vastas muscles are generally designated 114 and the remaining tissues are all collectively designated 116 and comprise the sartorius 123, rectus femoris 124 and tensor fascia lata 125 and vastus medialis 126. The first configuration may be defined by the relative arrangement of the various muscles and other tissues in the region.

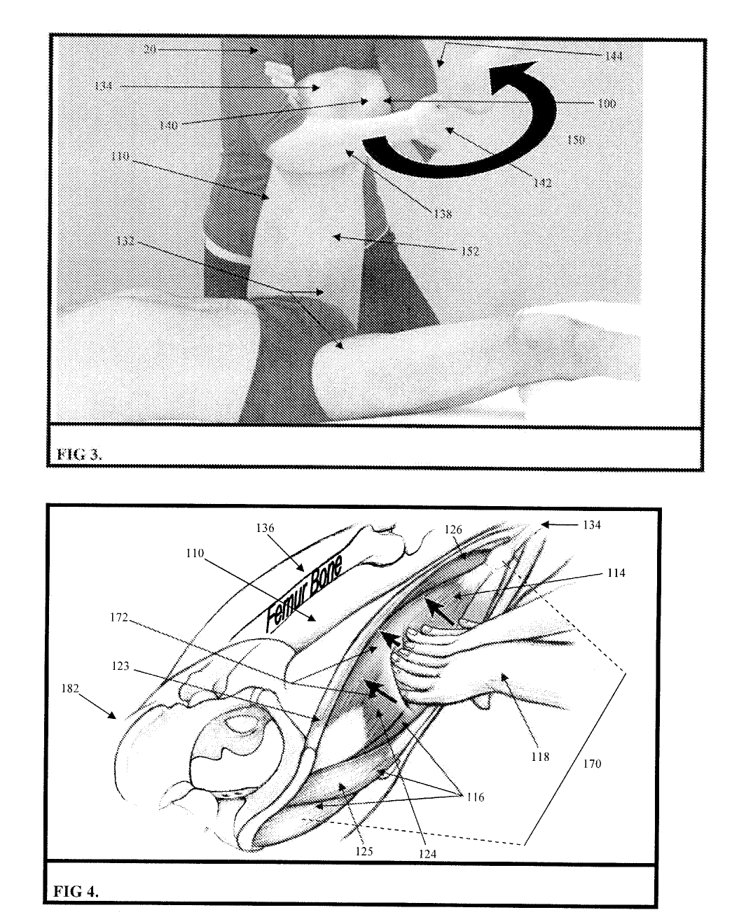

The method of the embodiment may comprise one or more of the steps of carrying out a first functional assessment as shown in FIG. 1. The tissue actuation may also be understood as a pattern of muscle loading. The assessment may be followed by carrying out an intervention. The intervention may comprise repositioning the first tissue in a second configuration to treat the musculoskeletal dysfunction. The repositioning is illustrated in FIGS. 4 and 5 where the first tissue is quad vastas complex 114 and is repositioned relative to other muscle groups and tissues 116.

The first functional assessment may comprise causing the mammal to actuate the body region in question, observing predetermined features of the actuation of the body region; and comparing those predetermined features during the observed actuation to the same predetermined features of a desired actuation to thereby assess the musculoskeletal dysfunction. The actuation may comprise holding the body region substantially static and functional assessment may further comprise observing the tissue actuation required to maintain stasis. The tissue actuation may also be understood as a pattern of muscle loading. Following an intervention changes in the pattern and distribution of loading may occur and may be observed as changes in the pattern of muscle loading.

Where the body region comprises the anterior leg, and in particular the anterior thigh 110 then as illustrated with reference to FIG. 1, then functional assessment may comprise causing the subject 10 to actuate the subjects muscles in a supine single leg raise at the hip 132 (with knee 134 maintained at full extension). The pre-determined features may comprise stability of the static limb, amplitude 120 of elevation, consistency of muscle or positional control, compensation in other muscle groups such as those in the trunk and body, and postural changes in the trunk and body and resistance to pressure applied by the practitioner. During this first functional assessment it may be seen that the leg in this position may have inconsistent control and limited resistance to a gentle hand force applied to the anterior ankle by the practitioner 20. The amplitude of elevation 120 may also be minimal.

In some embodiments, compensation for deficiencies in muscle functioning may be observed in the trunk 222 and upper body. The trunk 222 may become over-involved in raising the leg 100, such that axial rotation of the pelvis 184 and shoulders 200/202 may evident. The pelvis 184 may be ‘pouched’, or visibly deflected toward the table on the raised leg side.

The subject may be asked to make special note of the status of these features prior to treatment.

The body region may comprise a second tissue and the intervention to correct the musculoskeletal dysfunction may comprise repositioning the first tissue relative to this second tissue to establish a substantially stable second configuration of the musculoskeletal tissues. In FIGS. 4 and 5 the second tissue may be represented by any one or more of the muscle groups generally designated 116, which may comprise one or more of the rectus femoris, the sartorius, the tensor fascia lata, vastas, tibialis anterior, extensor hallucis longus. It will be understood that the terms first tissue and second tissue are not mutually exclusive and that in some embodiments the terms may overlap or that the first tissue may only be a different part of the same muscle, cartilage, tendon or other tissue comprised in the second tissue.

As may be best understood with reference to FIG. 3, repositioning may comprise positioning the subject 10 in supine position, such as on a suitable table 130, with hip 132 flexed to 90 degrees and knee 134 flexed to 90 degrees on one side. Practitioner 20 may interlock forearms 138 along medial aspect 140 of raised shank 143, keeping the lateral aspect 144 tight against the practitioners trunk. As practitioner gently rotates 150 towards the distal direction, the shank 143 may be abducted and the thigh 152 may be internally rotated.

For the second component of the treatment illustrated particularly with reference to FIG. 4, subject 10 is still supine, with practitioner 20 positioned ipsilateral to the side being worked on. The practitioner will work with both hands 160 together, or one hand over the rectus femoris (ipsilateral) and one hand under the biceps femoris. Starting at the knee 134, practitioner may internally rotate the first tissue, in this case the quadriceps 114, over the edge of a second tissue, in this case the femur of the same leg 100 (in the drawings the femur of the subject leg is hidden by muscle and other tissue but for reference the femur 136 of the other leg is shown). It will be appreciated that the first tissue, in this case the quadriceps, has a length 170 and in an embodiment the repositioning of the tissue comprises applying pressure in a direction generally indicated as 172 substantially perpendicular to the length 170. In the embodiment where the body region is the anterior leg 110 the Practitioner may achieve this by keeping application of force 172 perpendicular to the length of the femur 136 of the same leg 110. This manipulation should gradually progress in a proximal direction moving up towards the hip 132 to the tensor fascia lata. Here, manual force from the practitioner may be directed in a more anterior direction, still focusing on internal rotation of the muscle 114. Multiple passes of this manipulation may be beneficial, with the practitioner 20 focusing first on the relationships of rectus femoris and vastas lateralis, and on the sartorius (inner aspects of thigh) in the second set of manipulations as best shown in FIG. 5.

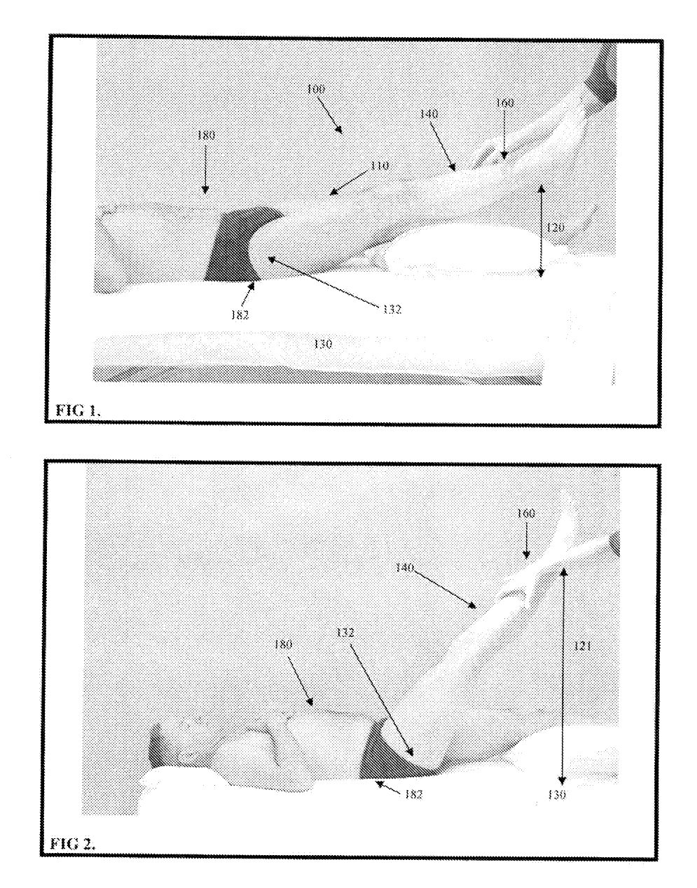

In some embodiments the method may further comprise conducting a second functional assessment best shown in FIG. 2. This second functional assessment may be carried out in substantially the same way as the first functional assessment. Where a second functional assessment is carried out the method may comprise comparing the properties of the predetermined features of the actuation observed during the first functional assessment with the properties of the predetermined features of the actuation observed during the second functional assessment. In embodiments the first and/or second assessments may comprise making measurements which may be noted or compared. When the intervention has been effective, substantial differences may be seen in the predetermined features between the first and second functional assessments. In the first embodiment, where the body region under examination is the anterior thigh 110, then after the intervention, it may be apparent that the amplitude 121 of elevation is substantially greater than amplitude 120 at the first functional assessment. The subjects resistance to force of hand 160 on the raised limb 100 may be substantially improved in magnitude. The trunk 180 and pelvic 184 regions may be less involved in the elevation, with the pelvis 184 and shoulders 200, 202 maintaining a neutral orientation, with symmetrical contact against the table 130. The subject may be asked to notice both the posture and control achieved in this second functional assessment, along with the correct muscle recruitment that achieves this control.

In further embodiments of the methods disclosed the method may comprise training the body region to habitually adopt the second configuration. This training may include muscle reeducation to maintain the new muscle configuration. This may comprise guiding a movement of the body region to follow a predetermined pattern while causing the mammal to concentrate on the quality of the guided movement The subject may be directed to practice suitable musculoskeletal motions to reinforce the new configuration. The subject may be directed to perform such practices daily and up to about 5, 10, 15, 20, 25 or more times each day, or each week, and for periods of up to about three, four, five, six, seven, eight, nine, ten or more weeks. Where the body region under consideration is the anterior leg the practice may comprise rolling the femur medially to bring the toes into a vertical position, and lifting the leg to about 45 degrees of hip flexion, noting the use of the quads and the position of the femur. The subject may also be directed to repeat these leg lifts 1, 2, 3, 4, 5, 6, 7, 8, 9, 10 or more times once a day for a 3-6 week period, and then occasionally thereafter. The subject may also be asked to roll the thigh muscles by hand medially 2-3 times a day for 3-6 weeks and thereafter occasionally if any discomfort is felt in the hips or lower back. Instruction of the subject may also comprise a demonstration of a suitable sitting configuration with the flesh of the seat of the body (primarily glutes) pulled laterally, to roll the femur medially. This sitting position may also help to maintain correct lumbar positioning. The subject may also be instructed to be mindful of maintaining a medially-rotated femur during walking and standing.

In some embodiments the method may have long term efficacy. In particular embodiments, once a 3 to 6 week course of treatment has been completed a subject may be substantially relieved of the musculoskeletal dysfunction and not require further visits to the practitioner.

In carrying out the first functional assessment, or the second functional assessment, or both, the subject may be urged to pay careful attention to their posture, muscle actuation, tissue configuration, tension and other aspects of their body functioning. The practitioner may draw the subject's attention to particular features or feelings associated with their body functioning, muscle configuration, tissue configuration, muscle actuation, posture, tension and the like during or after the assessment and again the practitioner may draw the patients attention to or urge the patient to pay attention to changes in these parameters. It will be understood that in particular variants any number of assessments, such as up to about 1, 2, 3, 4, 5, 6, 7, 8, 9, 10, 11, 12 or more assessments may be carried out. A plurality of such assessments may be spread out over extended periods in separate treatment sessions that may be separated by about 1, 2, 3, 4, 5, 6, 7, 8, 9, 10 or more days, or by 1, 2, 3, 4, 5 or more weeks or by 1, 2, 3, 4, 5 or more months. A plurality of assessments may be comprised in an individual treatment session or spread out over multiple sessions and may relate to the same or different body regions.

It will be understood that although in the embodiments described force may be exerted or evaluated through or by any part of the body of a practitioner, in alternative embodiments such force may be exerted or evaluated using artificial aids or devices which may or may not be controlled or guided by a human subject.

In particular alternative embodiments different body regions may be evaluated and/or treated in a predetermined sequence. It one alternative embodiment it may be found most effective to address the muscles of the anterior body first. It may be found that treating the posterior of the body is less effective that when the anterior is treated first it becomes more repetitious or problematic to accomplish all the necessary alignments effectively.

For example, in alternative embodiments a number of particular embodiments illustrated by the Examples may be combined into a sequence, one possible sequence for treatments or evaluations may be or may comprise the following sequence of steps: (i) lower anterior abdomen; (ii) shoulder girdle; (iii) forearm; (iv) neck; (v) anterior legs, (vi) lower back and posterior pelvis, (vii) posterior leg, (viii) mid back and posterior shoulder, (ix) core axis, (x) anterior shoulder and upper trunk. In alternative embodiments additional body regions may be included in this sequence of steps, and this sequence of steps may be amended, in ways that will be readily understood by those skilled in the art.

It will be understood by those skilled in the art that the functional assessments may comprise the use of the apparatus and methods described in more detail in connection with the second embodiment hereof.

### Second Embodiment

In a second embodiment the methods and approaches described in relation to the first embodiment may be used to evaluate a musculoskeletal dysfunction in a body region of a mammal, which again may be a human. Again the body region may have a tissue configuration and the method may comprise causing the subject human to actuate the body region; observing predetermined features of the actuation of the body region; and comparing those predetermined features of the observed actuation to predetermined features of a desired actuation to evaluate the musculoskeletal dysfunction.

As again illustrated with reference to FIG. 1, the method may comprise causing the mammal in this case a human 10, to actuate the body region in question, observing predetermined features of the actuation of the body region; and comparing those predetermined features during the observed actuation to the same predetermined features of a desired actuation to thereby assess or otherwise evaluate the musculoskeletal dysfunction. The actuation may comprise holding the body region substantially static and functional assessment may further comprise observing the tissue actuation required to maintain stasis. The tissue actuation may also be understood as a pattern of muscle loading.

In some embodiments, compensation for deficiencies in muscle functioning may establish a recognised pattern of recruitment of compensation that may be observed in the trunk 222 and upper body. The trunk 222 may become over-involved in raising the leg 100, such that axial rotation of the pelvis 184 and shoulders 200, 202 may evident. The pelvis 184 may be ‘pouched’, or visibly deflected toward the table on the raised leg side. The elevation 120 of limb 100 may be substantially reduced compared to a desired or normal elevation 121 as shown for comparison in FIG. 2.

The carrying out of such evaluation may not require the repositioning step, and may not require that a second functional assessment be carried out. A range of alternative forms of the second embodiment may be possible in the same way as described in relation to the first embodiment. It will be further understood that in some embodiments the evaluation may comprise carrying out a repositioning step as set out in relation to the first embodiment, and observing the effects of the repositioning on particular predetermined parameters of the actuation of the body region. Such further observing may comprise the use of an apparatus 490 which may be suitably programmed.

In alternatives, the assessment will or may comprise the use of an apparatus generally designated 490 and illustrated in FIG. 50. This may comprise one or more sensing elements 500, connected to a processor 510 by wires 520 which may be replaced by a wireless connection or any other suitable means for the sensing elements 500 to communicate with processor 510. Sensing elements 500 are positioned at suitable desired locations on the body of subject 10. Processor 510 is programmed using conventional methods to display on a display or monitor 530, information regarding properties that may comprise one or more of the electrical activity, contraction and position of muscles in and associated with the body region under investigation and the position of the body region, in this case leg 100.

In particular embodiments the apparatus may determine the existence and properties of any improvement between different assessments or before and after any intervention. The apparatus may comprise a display or monitor or may be suitable to generate a printout or other hard copy. The results generated by the apparatus may comprise information on the pre intervention and/or post intervention actuation and may be useable by a practitioner, to assist in evaluating a musculoskeletal dysfunction.

In alternative embodiments the sensing elements or processor of the apparatus may be or may comprise one or more types or models of device suitable for deriving analyzing or displaying data on muscle actuation, body region or tissue configuration etc. By way of example they may include commercially available devices, software or systems or parts thereof and may include products such as CT scanners, MRI scanners, P.R.O.O.F. assessment packages, MICROFET devices, such as MICROFET 3MT & ROM, inclinometers, muscle testers, range of motion monitors (such as cervical range of motion instruments and back range of motion instruments), sitting and flexibility testers, and other measuring devices. In alternative embodiments they may accept or process data derived from such devices. Any such devices or data therefrom may be useable in certain embodiments to construct a single apparatus or appliance. In particular embodiments the apparatus and method may comprise 2D or 3D displays of data and may include modeling software to generate 2D or 3D representations of data or of muscle actuation to assist interpretation by a user. A range of suitable devices and systems may include those manufactured by HOGGAN HEALTH INDUSTRIES, or aspects thereof, but a variety of alternative devices will be readily apparent to those skilled in the art who will readily understand their use and application and will be able to make any necessary adaptations for their use in the embodiments describe herein. It will be further understood that in particular embodiments the apparatus and systems used may comprise memory systems or stored data to allow comparison with known patterns of actuation and configuration in order to assist a user to better evaluate data.

In particular embodiments the input from one or more devices may be combined and the data may be processed to generate images of the subject as they present at different time points. The apparatus may be used to assist the patient to notice changes resulting from repositioning steps and to assist the practitioner to monitor progress. The apparatus may also be interconnectable to take account of medications, radiological and other relevant medical data. The apparatus may be configurable to allow a plurality of practitioners, optionally from a plurality of disciplines, to provide input to a single patient chart. In embodiments the apparatus, system, software, or other aspects of embodiments may be useable by practitioners, including sports medicine practitioners, to evaluate fitness levels, fitness to perform, or recovery from injury.

### Third Embodiment

In a third embodiment there is described a method for enhancing the performance of a body region of a mammal, which may be a human. This third embodiment may comprise essentially the same elements as the first embodiment. The only difference being that in this third embodiment the functional assessment or assessments and any intervention may be conducted not to overcome a dysfunction, but to improve the performance of a body region that is functioning in a manner that while acceptable by normal standards, may be capable of improvement by the repositioning of particular tissues.

### Fourth Embodiment

In a fourth embodiment there is described a method for training others to use a guide to treat a musculoskeletal dysfunction in a body region of a mammal which may be a human having first and second tissues. In certain respects this fourth embodiment may comprise essentially the same elements as the first embodiment. However, in this embodiment the functional assessment or assessments and any intervention may be described in a guide which can be used a training tool to carry out the steps of training others to carry out one or more functional assessments as generally described for the other embodiments and make any necessary or desirable comparisons of the predetermined features either between particular assessments or between particular assessments and predetermined model data or both. The guide may also be useable in training others to use the guide to correctly reposition the first tissue in a second configuration to treat the musculoskeletal dysfunction.

### EXAMPLES

The following examples are presented in the form of specific alternatives, examples and variants of the first embodiment. Those skilled in the art will understand that all the examples or elements thereof may also be realised in the form of a diagnostic method according to the second embodiment or in the form of a method for enhancing performance according to a third embodiment and accordingly these Examples or parts thereof may be considered additional examples of all of the first three embodiments and may in particular alternatives be implemented using the methods and apparatus comprised in each of the embodiments.

### Example 1

Lower back and posterior pelvis: The muscles of the lower back provide for stability of the spinal column and inter-segmental trunk movements (rotation, lateral flexion, extension), while the muscles of the posterior pelvis stabilize the head of the femur. This functional group includes the quadratus lumborum, the multifidus, and the piriformis. Functional deficits in this group typically result from either acute injury, cumulative loading of the soft tissues of the low back 192 through repetitive, awkward, or static action of the trunk, or due to chronic lateral deflection of the femur and the opposing supportive psoas major. This lateral deflection may lead to over-recruitment of the quadratus lumborum, multifidus, and piriformis during lifting movements and other trunk actions. In all cases, the tissues of the lower back and posterior pelvis 194 may be tense and sensitive to the touch, with a hard, ropy nature and limited pliability. This deficit tissue status may also lead to increased compressive loading on the intervertebral discs of the lumbar spine, which may be a precursor to a wide array of chronic musculoskeletal problems.

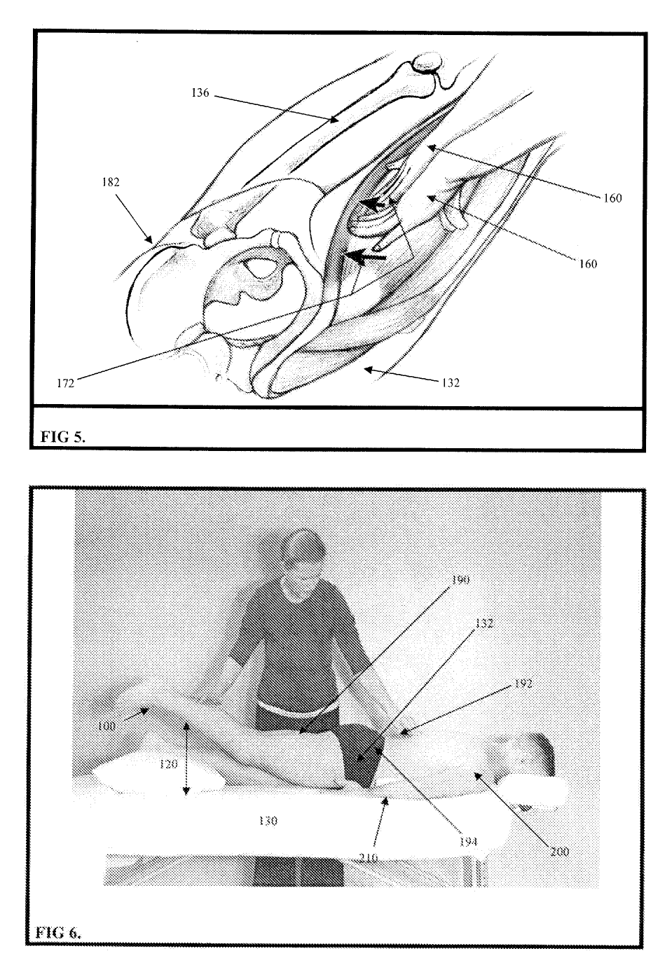

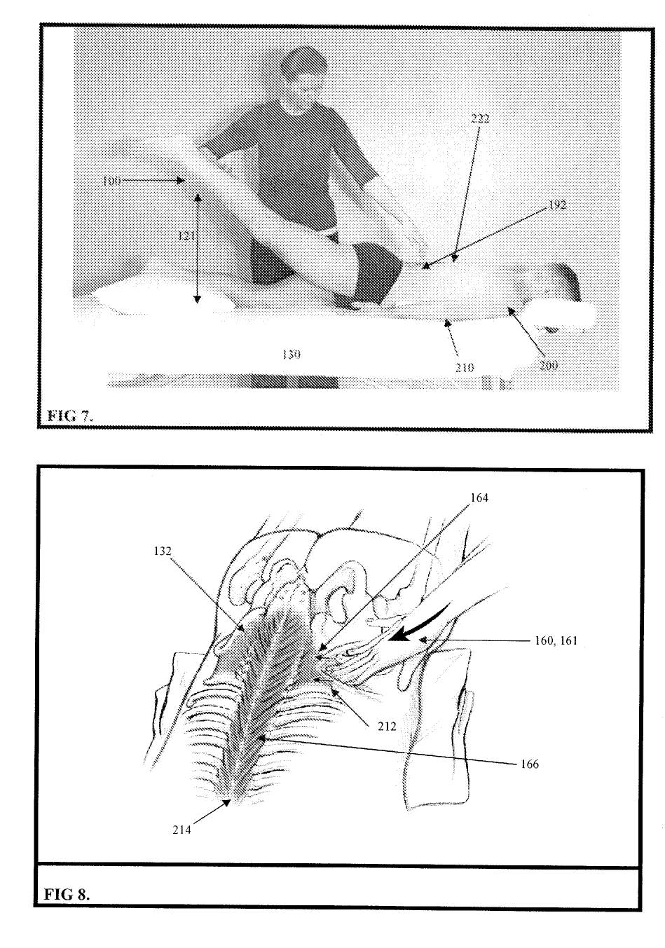

Functional assessment—pre and post intervention: The functional assessment for this group is best seen in FIGS. 6 and 7, a prone single leg raise (hip extension). Pre-intervention, as best shown in FIG. 6, the leg raise will be of minimal amplitude, with inconsistent control and limited resistance to fingertip pressure applied to the calf by the practitioner. Subjects who have been reporting lumbar pain prior to the intervention may experience discomfort when attempting to lift the leg 100. In these cases, specific respect for the subject's limitations must be given.

Compensation may also be observed in the shoulders 200, 202, arms 210, and lower back. Specifically, the trunk 180 may become over-involved in raising and balancing the limb, such that exaggerated lumbar lordosis and elevation of the shoulders is evident. The subject should make special note of these challenges.

The subjects resistance to fingertip pressure on the raised limb 100 should be dramatically improved in magnitude and consistency following intervention, and the amplitude of leg raise 120/121 achieved should increase. Subjects who reported pain in the pre-intervention functional assessment should also note a sharp decrease in pain. In parallel, less involvement of the trunk should be observed, with the lumbar region maintaining its moderate lordosis, the shoulders 200,202 remaining in contact with the table 130, and no recruitment of the arms 210 for counter-balance. Again, the subject needs to make mental note of both the posture and control achieved in this assessment, along with the muscle activation that provides this control.

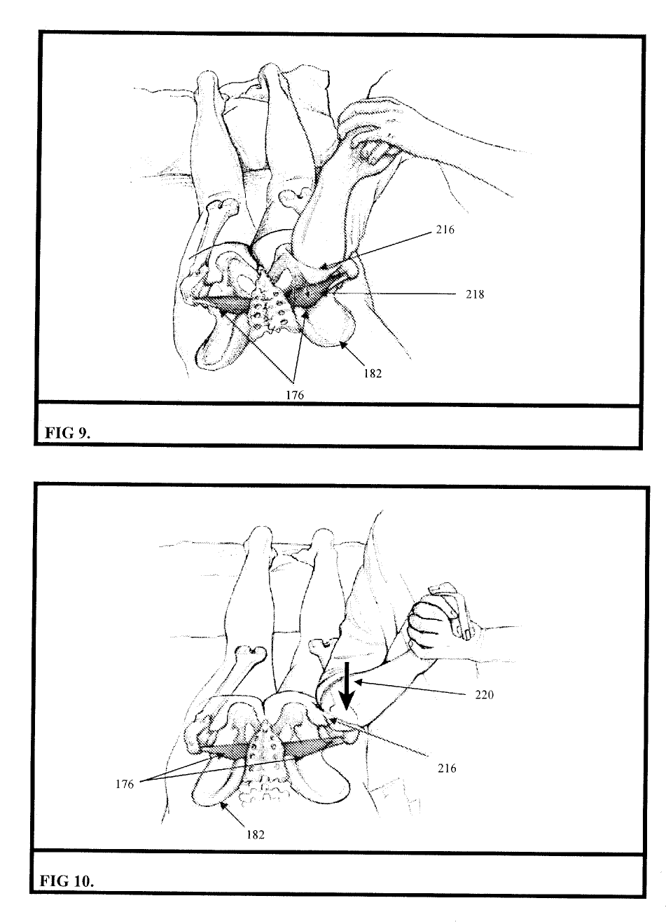

Intervention: With subject 10 prone on the table 130, practitioner 20 is positioned ipsilateral to the side being worked on. As shown in FIG. 8. The practitioner will work with the finger tips of both hands 160 together, applying pressure to the lateral edge of the quadratus lumborum 164 The practitioner then applies force 212 perpendicular to the length of the quadratus lumborum 164, adjusting it back toward the mid-line of the body. This perpendicular force application starts at the T12 level of the quadratus lumborum 164, and proceeds stepwise down to the lowest levels of the muscle at the hip 132. Multiple passes may be required on each side to provide functional improvement. This treatment also improves the alignment and activation of the multifidus 166 as it is caught up in the perpendicular realignment toward the spine 214.

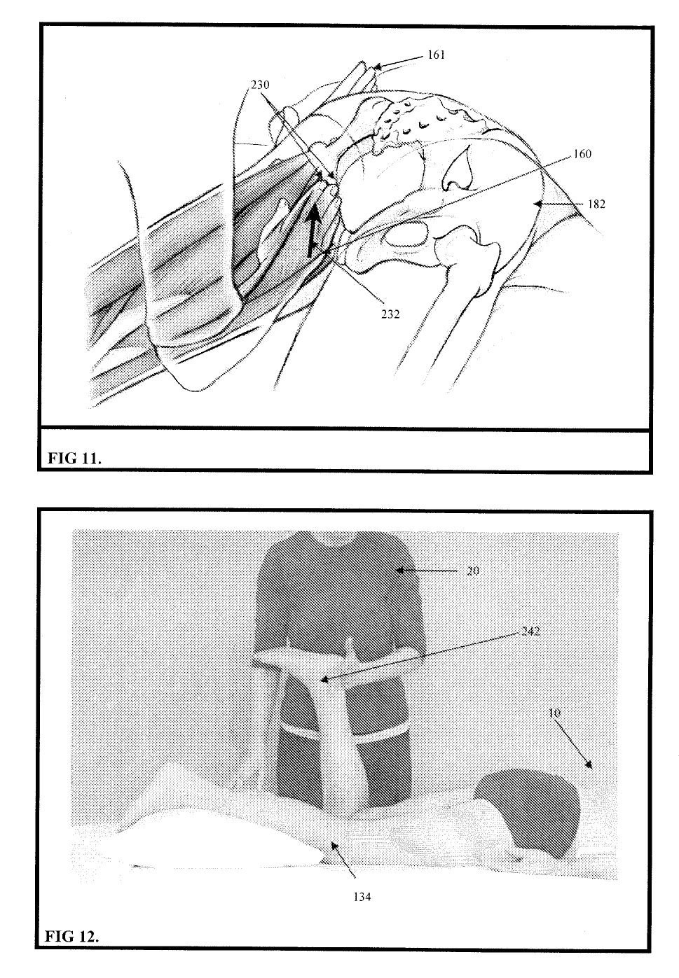

The second component of the treatment is illustrated with reference to FIG. 9, subject 10 is still prone. The practitioner 20 uses their elbow 216 to apply pressure in a semi-circular pattern 218 to the lateral and posterior aspects of the gluteus maximus in the region of the deeper piriformis, and it's connection to the greater trochanter. The point pressure of the elbow 216 allows the force to penetrate the gluteus maximus and release tension in the piriformis 176. Again, this manipulation needs to be performed on both sides of the body.

The third component of this treatment is shown with reference to FIG. 10 and comprises an acute release of the iliopsoas connection, returning it to a more medial (and functional) position. In this manipulation, the provider produces a downward thrust 220 of their elbow 216, which is positioned perpendicular and superior to the subject's trochanteric bursa area in the prone position.

Occasionally, a subject's problems with lifting their leg may persist, most noticeably compensation in the hip 132 and trunk 222, even after the previous outlined steps are taken. In these cases, the practitioner may continue with treatment by laterally deflecting the leg 100 of the side being worked on. As best illustrated with reference to FIG. 11, this positioning allows the practitioner to place two firmly extended digits 230 of the dominant hand 160 onto the insertion point of the psoas major, accessing it from the posterior side of the body through the caudal groin. The practitioners opposite 161 hand is pressed into the outer hip region for counter balance, while the practitioner thrusts pressure in an upward, diagonal and lateral direction 232 to realign the psoas major. The practitioner then brings the subject's leg 100 back to center, and asks the subject to repeat the leg lift. Generally there is more strength with less compensation. This may need to be repeated 2 or 3 times in the initial treatment with improvement noted by both practitioner and subject with each repetition.

### Example 2

Posterior leg: The muscles of the posterior leg 190 provide for extension and adduction at the hip 132, flexion at the knee 134, and plantar flexion of the foot 242. This functional group includes the gracilis, the semimebranosus, the biceps femoris (long and short heads), the gastrocnemius 188 and the soleus 189 all bilaterally. Functional deficits in this group may be the result of excessive external rotation of the lower limbs during walking, stair climbing, and quiet standing, as well as in seated, prone, and supine resting postures. Muscles and soft tissues may deflect in the lateral direction as a result of this chronic loading, leading to weak muscle force production and reductions in key parameters of gait, including stride length and rate. In addition, small areas of muscle tightness are frequently found between the two heads of the gastrocnemius. 188 These nodes are highly sensitive to pressure on the overlying skin surface, and are a consistent finding among subjects with problems in the lumbar and sacroiliac regions. It is possible these nodes are further manifestation of sciatic impingement from protruding intervertebral discs.

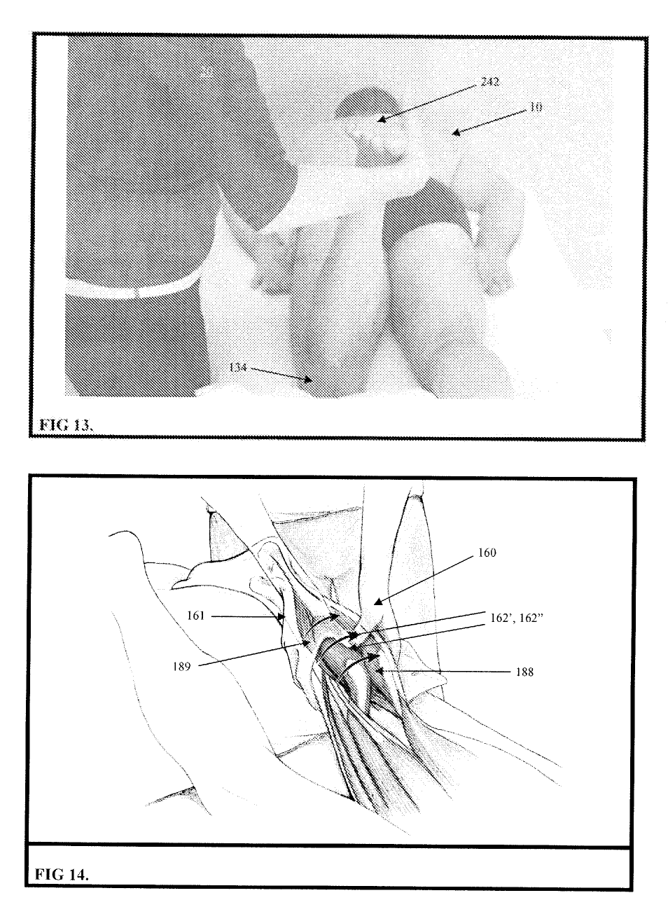

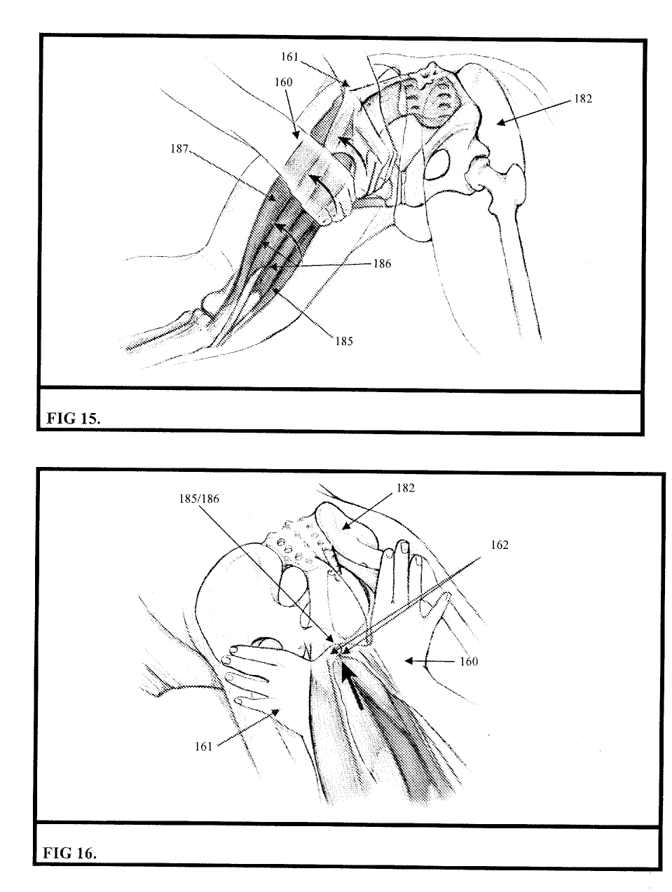

Functional assessment: Pre- and Post-intervention: There are two functional assessments for this group in perpendicular directions, though each is a prone single leg action with the knee flexed at 90°. In the first assessment shown in FIG. 12, resistive force from the practitioner is applied in the distal direction on the subjects heel. In the second assessment as shown in FIG. 13, the practitioner applies resistive force in the lateral direction to the inside aspect of the subject's raised ankle. Pre-intervention, the shank in this position will have inconsistent control and limited resistance to hand resistance applied to the heel by the practitioner in either direction. In addition, compensation can be observed in the trunk and upper body. Specifically, the trunk will become over-involved in elevating the limb 100, such that axial rotation of the pelvis 184 and shoulders 200, 202 is evident. The subject should make special note of these challenges prior to the treatment.

Following treatment, the subject's resistance to hand resistance on the raised limb should be dramatically improved in magnitude. The trunk 222 and pelvis 184 should also be less involved in the elevation, with both the anterior hips 132 and shoulders 200, 202 remaining in contact with the table 130. Again, the subject should be instructed to make mental note of both the posture and control achieved in this second functional assessment, along with the muscle activation that provides this control.

Intervention: As best seen in FIG. 14 with subject 10 prone on the table 130, practitioner 20 is positioned ipsilateral to the side being worked on. The practitioner will work with both hands 160 cupped and pressed together at the thumbs 162 to create a single manual unit. The practitioner starts at the proximal end and applies pressure to the medial edge of the gastro/soleus complex, deflecting the whole complex laterally while progressively moving in the distal direction.

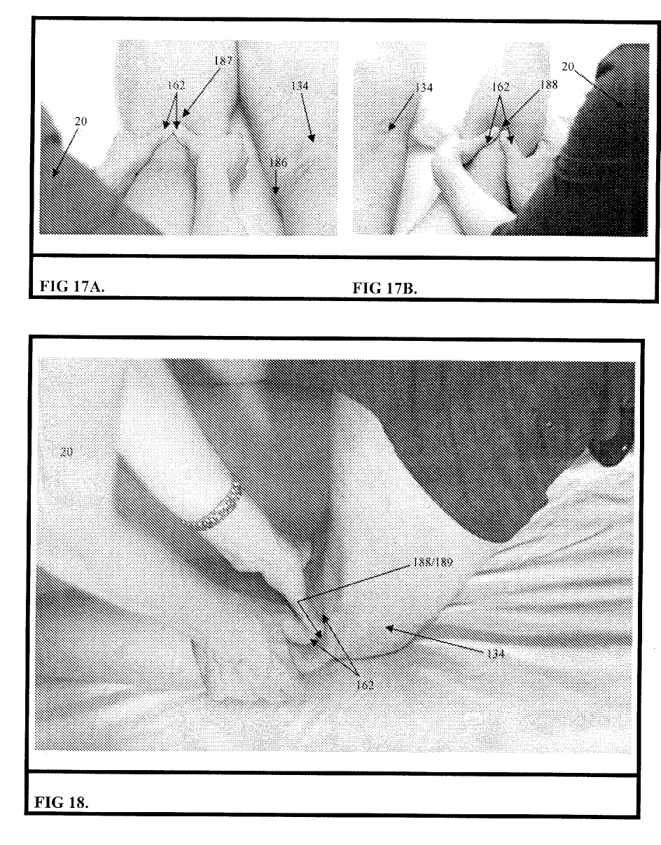

For the second component illustrated with reference to FIG. 15 of the treatment subject 10 is prone. The practitioner 20 maintains the hands 160 together curled finger position, and manipulates gracilis 185, semimembranosus 186, and biceps femoris 187 in the lateral direction, starting at the distal end of the group and progressing in the proximal direction to the level of the glutes. The femur can be used as counter-pressure point for this action, allowing for reactive external force application to both the anterior and posterior surfaces of these muscles. This component ends with a release of the semimembranous 186 and gracilis 185 at their proximal attachments. Finally, as shown in FIG. 16 the practitioner uses both thumbs 162 to apply pressure at the insertion point of the semimembranous and gracilis with a thrust directed cranially.

In the third component of this treatment, subject 10 is still prone. Practitioner 20 places thumbs 162 of both hands on the posterior proximal aspect of the knee joint 134, with the thumbs 162 on the distal aspects of the semimembranosus 186 and biceps femoris 187. The practitioners finger tips 163 should be positioned along the medial and lateral aspects of the knee 134 as appropriate. The practitioner will apply a vigorous pressure with the thumbs 162, with the force directed in an anterior and proximal direction, best seen in FIG. 17A. A similar manipulation is applied to the distal component of the posterior knee joint, where the practitioner applies firm force, through their thumbs, to the proximal ends of the gastrocnemius 188 muscle and is shown in FIG. 17B. In this application, the direction of force should be anterior and distal, with the thumbs also moving from a central position to medial and lateral positions as the action progresses. This pair of actions serves to reduce compression or other stress in the knee joint.

### Functional Assessment and Intervention for Limited Knee Motion

The final component of this section is used if the subject is experiencing knee compression pain or binding on the anterior side or on the lateral side of the knee joint 134, along with limited range of knee motion. The subject 10 is asked to kneel on the table, with forearms 244 also resting on the table. As a first functional assessment, the subject is asked to flex the hips 132 and knees 134 in this kneeling position, bringing the seat back to the feet 242. Pain or binding during this assessment, along with a limited range of motion, indicates a need for the intervention.

For this intervention, illustrated with reference to FIG. 18 subject 10 remains in the kneeling position with hips 132 just anterior to knees 134. Practitioner is positioned ipsilateral to the limb 100 being worked on. This alignment can be acutely uncomfortable, so the subject is warned to expect a moment of pain and to breathe deeply and slowly while the intervention is progressing. The practitioner places thumbs 162 in a deep pressure at the proximal endpoint of the soleus 189 and gastrocnemius 188 muscles, and rolls these muscles to a more lateral position. In the second functional assessment, the subject is asked to repeat the first functional assessment and compare the experience. When the alignment is complete the subject's range of motion during the assessment will be increased, and the subject will experience less compression with little or no pain. If the subject's condition is improved but still presents problems, repeat the alignment and repeat the second functional assessment until desired results are achieved.

### Example 3

Muscles of the mid-back and posterior shoulder: The muscles of the mid back provide for inter-segmental trunk movements (extensions and static flexed trunk postures), while the muscles of the posterior shoulder move the scapulae. Due to the large range of motion at the shoulder, necessary movements for the scapula include elevation, rotation, retraction toward the posterior mid-line, and depression toward the central mid-point. This functional group includes the erector spinae, all three aspects of the trapezius (superior, middle, and inferior), the levator scapulae, and the major and minor rhomboids. Functional deficits in this group are the result of chronic and progressive lateral deflection of segments and supportive soft tissues, due to the highest relative frequency of flexion at the shoulder and trunk, a cumulative result of the many activities of daily living that occur anterior to the mid-line of the body. In all cases, the tissues in question can be tense and sensitive to the touch, with a hard, ropy nature and limited pliability. Specific signs and symptoms from deficits in this area may include muscle spasms in the thoracic region, muscular tension leading to severe headaches, poor posture leading to limitations in deep breathing, and chronic neck and shoulder pain. Weakness and misalignment in the thoracic region can also lead to aggravation of various spinal deformities, including osteoarthritis and scoliosis.

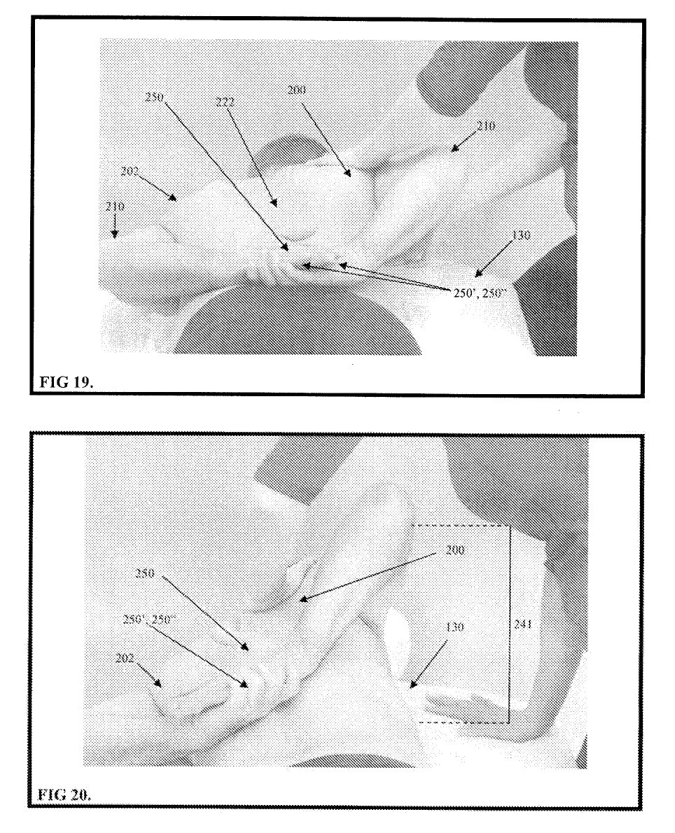

Functional assessment: Pre- and Post-intervention: The functional assessment for this group illustrated with reference to FIG. 19 is a prone posterior deflection of both arms 210, such that the hands 250 meet as high as possible on the subjects back. This action is combined with a posterior deflection of one shoulder 200, to maximum possible amplitude. Pre-intervention, the posterior shoulder raise will be of minimal amplitude, with inconsistent control and limited resistance to fingertip pressure applied to the back of the shoulder 200 by the practitioner 20. This limited resistance can be characterized as a ‘rocking’ off the torso around the axial midline. Compensation can be observed in the contralateral shoulder 202 and trunk 222. Specifically, trunk 222 will become over-involved in raising the shoulder 200, such that the contralateral shoulder 202 is more firmly pressed against the therapy table 130. In addition, the subject may display difficulties in bringing the hands together in the back during the pre-check, and the position of the hands will be close to the lumbar region. The subject may also report symptoms of pain during this action. The subject should make note of these challenges.

As illustrated in FIG. 20, this subject's resistance to light physical pressure from the therapist should improve in magnitude and consistency following intervention, and the amplitude of raise achieved should increase. Less involvement of the trunk should be observed, with the contralateral shoulder 202 remaining in comfortable contact with the table 130. Finally, the hands 250 should easily meet in the back, and their location should be in the thoracic region of the dorsal surface. Again, the subject 10 should make note of the improvement here, with focus on the increased amplitude 241 of shoulder lift, the more superior position of the crossed hands, and the primary muscles activated to reach this new position.

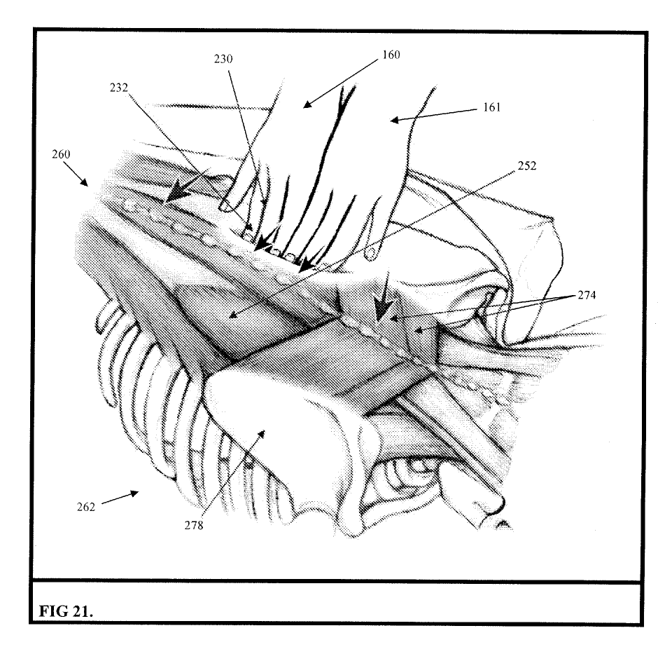

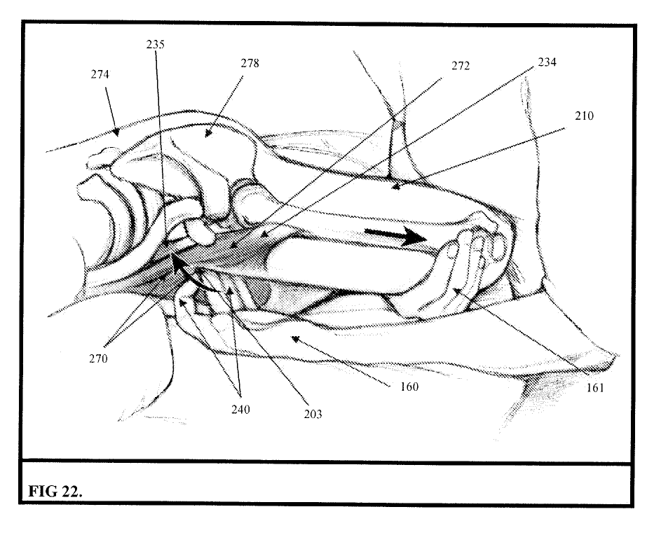

Intervention: As illustrated with reference to FIG. 21, with subject 10 prone on the table 130, practitioner 20 is positioned ipsilateral to the side being worked on. The practitioner 20 will start with pressure from both thumbs 162 between each spinous process, starting at C7. The practitioner then works with the finger tips 230 of both hands together, applying pressure to the lateral edge of the erector spinae 252. The practitioner then applies force 232 perpendicular to the length of the erector spinae 252, adjusting it back toward the mid-line 260 of the body. This perpendicular force application starts at the T12 level of the erector spinae 252, and proceeds cranially to more superior levels of the muscle in the upper back (generally designated 262). Multiple passes may be required on each side to provide functional improvement—the muscles can typically be deflected further in the medial direction on each subsequent pass.

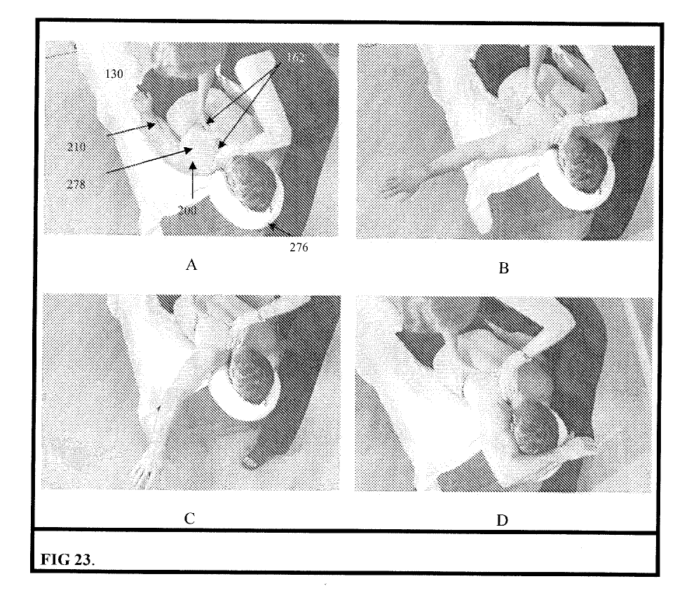

For the second component of the treatment, shown in FIG. 22, subject 10 is still prone with their arm 210 resting at the side of the body. The subject must remain passive during this adjustment, even though there may be some mild to acute pain if the muscles have been in misalignment for a period of time. The practitioner 20 is seated on the subject's ipsilateral side, with middle fingers 240 curled along the underside (caudal edge) of the pectoralis major 203. The practitioner applies force 270 perpendicular to the orientation of the pectoralis muscles 272, pulling the muscles in a cranial direction while working the fingers 240 toward the insertion of the pectoralis muscle. This alignment helps to release the full shoulder, and the rhomboids 274, for the subsequent treatment, but it may take two or three passes to totally release the rhomboid. Both the subject and practitioner will note the changes in the tension of the rhomboid while working.

The third component of this treatment is mobilization of the scapula and alignment of the rhomboids 274 as illustrated with reference with FIG. 23 A-D. The subject places the arm 210 overhead onto the face rest 276, and tracks that arm through shoulder abduction/adduction while the practitioners hands 160, 161 are positioned surrounding the edge of the scapula 278, with a thumb 162 from each hand above and below the rhomboid applying deep pressure. As the subject tracks the arm 210 the practitioner stretches the rhomboid muscle in the opposite direction the arm is being tracked. This combined movement of both subject and practitioner assists in the scapula 278 moving more freely.

### Example 4

Soft tissues of the core axis: The soft tissues of the core axis are responsible for the maintenance of our erect posture, as well as basic control of the functional movements, in all three axes, that originate from our axial skeleton foundation. This functional group can be subdivided by tissue type (ligaments and muscles) and anatomical zone (anterior and posterior). Posterior ligaments of the core axis include costotransverse, supraspinous, and iliolumbar ligaments, as well as the articular capsules, while the major ligament of the anterior core axis is the anteriorlongitudinal ligament. The muscles of the posterior core axis include the inter- and semispinalis, the spinalis, the splenius, the rotator longi and brevi, the intercostals, and the multifidis, while the muscles isolated to the anterior core axis group for this treatment are the intercostals and the multiple layers of the obliques. Functional deficits in this group are the result of chronic and progressive anterior and lateral deflection of segments and supportive soft tissues. In part, this deflection results from non-neutral postures. In addition, our frequent shoulder and trunk flexion actions necessitated by our targeted movements to the anterior and lateral regions around the body lead to this deflection.

This progressive deflection leads to a coupled response in our soft tissues of the core axis. Soft tissues on the posterior side of the body are under chronic tension in this model, leading to a progressive reduction in tissue properties, including strength, optimal orientation, and response rate. On the anterior side of the body, soft tissues are in compression during these non-neutral postures. Chronic compression of soft tissues can also limit the natural tissue properties, including firing rate and strength.

Functional assessment: Pre- and Post-intervention: The functional assessment for this group is a prone posterior deflection of both arms, such that the hands 250 meet as high as possible on the subject's back. This action is combined with a posterior deflection of one shoulder, to maximum possible amplitude. Pre-intervention, the posterior shoulder raise will be of minimal amplitude, with inconsistent control and limited resistance to fingertip pressure applied to the back of the shoulder by the practitioner. Compensation can be observed in the contralateral shoulder 202 and trunk. Specifically, trunk will become over-involved in raising the shoulder, such that the contralateral shoulder 202 is more firmly pressed against the therapy table. In addition, the subject may display difficulties in touching hands in the back during the pre-check, and the position of the hands will be close to the lumbar region. The subject should make note of these challenges.

This subject's resistance to light physical pressure from the practitioner should improve in magnitude and consistency following intervention, and the amplitude of shoulder raise achieved should increase. Less involvement of the trunk should be observed, with the contralateral ‘shoulder 202 remaining in comfortable contact with the table 130. Finally, the hands should easily meet in the back, and their location should be in the thoracic region of the dorsal surface. Again, the subject should make note of the improvement here, with focus on the increased amplitude of shoulder lift, the more superior position of the crossed hands, and the primary muscles activated to reach this new position.

Intervention: The practitioner 20 stands parallel to the prone subject 10, contralateral to the shoulder 200 being manipulated. The practitioner 20 will lean over the subject 10 with thighs braced against the table for balance, and lace their hands under the subject's raised shoulder 200, providing comfortable resistance. The subject will exhale through the mouth, then push forward (into the hands of the practitioner) to full range, ending with relaxation. In all cases, the subject needs to focus on pushing as a motion that involves the core axis and the full thorax, not the shoulder and arm. The subject also needs to focus on maintaining a neutral head and neck posture during these activations.

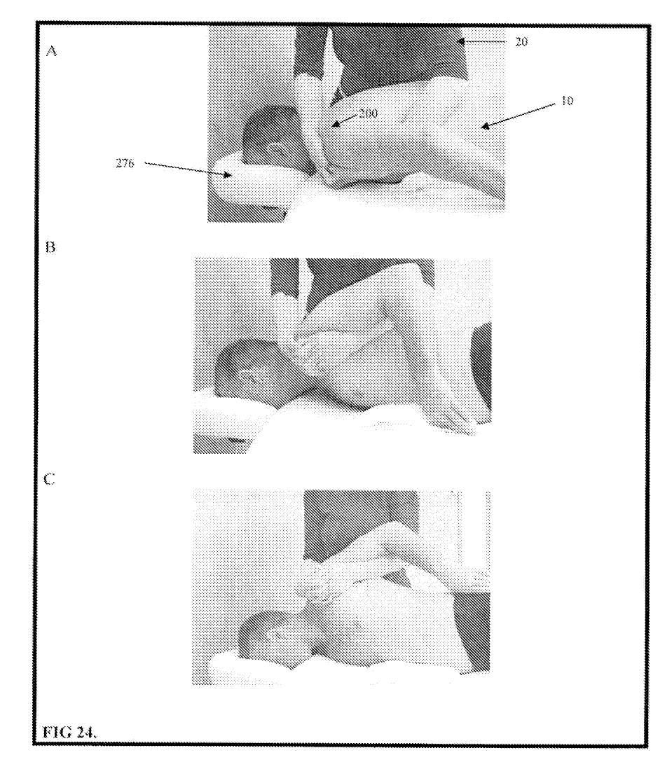

As illustrated in FIG. 24 A-C, when the subject 10 relaxes subsequent to each exertion, the practitioner 20 will increase the resistant range by pulling further back on the shoulder. This treatment can progress through three iterations, with the practitioner providing increased resistant range in each trial. To increase the extension of core axial tissues in the cervical region, the practitioner can place a single hand under the ipsilateral 200 and a second hand on the back of the head, with the thumb in the ipsilateral occiput. The subject should inhale prior to this manipulation. As the subject exhales and relaxes, the practitioner will stretch the space between the two hands as shown in FIG. 25.

### Example 5

Muscles of the anterior shoulder and upper trunk: The major muscles of the upper arm and shoulder include the three heads of the triceps brachii the coracobrachialis, the serratus anterior, the deltoid, the subscapularis, and the pectoralis major and minor. Functional deficits in this group are the result of acute injury, or chronic and progressive lateral deflection of segments and supportive soft tissues. In part, this deflection results from non-neutral postures. Frequent shoulder and trunk flexion actions in the anterior and mid-sagittal zones of the body lead to this progressive deflection.

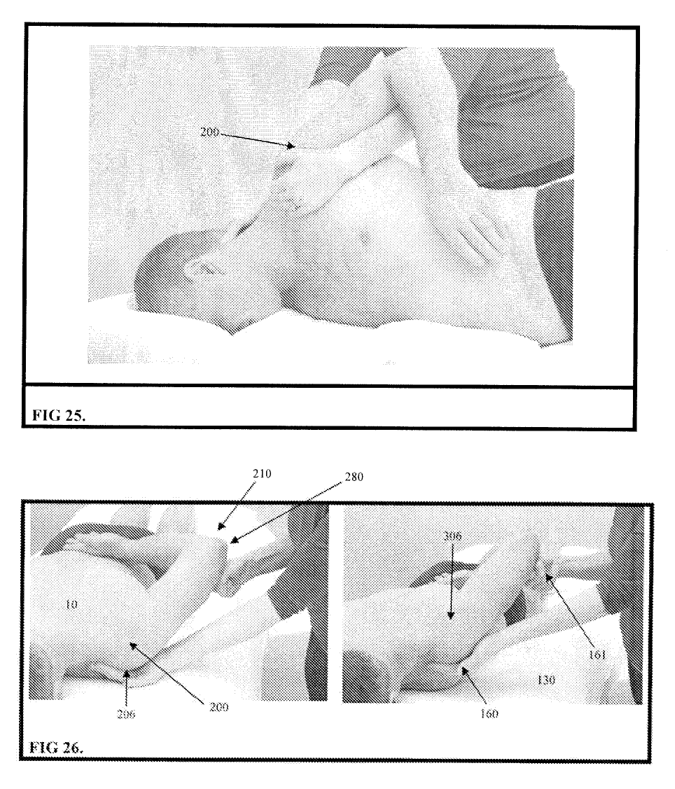

Functional assessment: Pre- and Post-intervention: There are two functional assessments for this group which are illustrated with reference to FIG. 26. The first is a prone posterior deflection of one arm 210, with the elbow 280 flexed at 90° with the wrist 282 at the posterior waistline (lumbar area). In some cases, the subject 10 maybe unable to reach this initial 90° position, due to tension and/or binding in the coracobrachialis 206 Attempts to further flex the elbow 280 for these subjects may produce pain. For subjects who can achieve this starting position, most pre-intervention participants will be unable to produce adequate force, and may experience pain in the attempt to activate the bent arm in the anterior direction with the practitioner offering resistance. This activation isolates the coracobrachiali 206. Practitioner 20 should perform this assessment at two different amplitudes, as the force generated can vary greatly in different orientations. It is important that the subject notice their limitations in this position, along with any discomfort.

In the second functional test, with the arm in the same position, tricep 306 weakness is evidenced as an inability to produce adequate force in a posterior direction against resistance from the therapist on the posterior side of the same bent arm. In trying to produce this resistance, pre-intervention subjects will recruit the latissimus dorsi and erector spinae on the contralateral side. It is important that the subject notice their limitations in this position, along with any discomfort. Following treatment, the subject should be able to produce suitable force to oppose resistance at the elbow from either direction while the arm is bent behind the back. Again, the subject should notice this improved force production, along with the muscle activation that provides it.

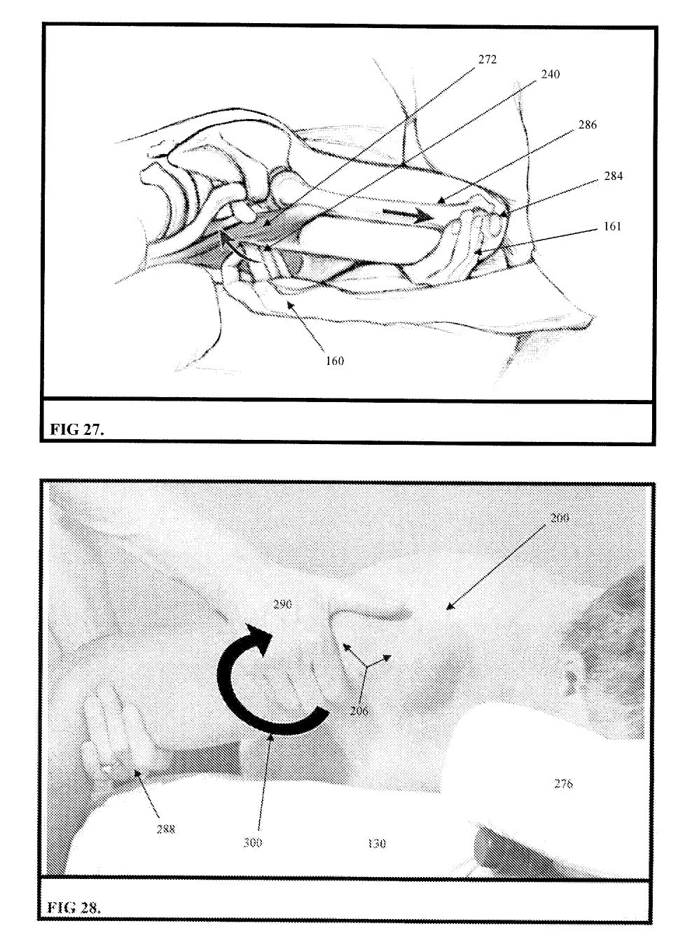

Intervention: As illustrated with reference to FIG. 27-30, with subject 10 prone on the table 130, practitioner 20 is positioned ipsilateral to the side being worked on. The practitioner will hook three fingers 240 of the dominant hand 160 to the clavicle origin on the lower side of the pectoralis 272 muscle, deflecting this muscle in the cranial direction (as seen in FIG. 27). In addition, tension is released from the soft tissues of the pectoralis 272 by cupping the free hand 161 around the distal end 284 of the humerus 286 and gently dislocating the upper arm in a lateral direction. Following the alignment, the subject will return the arm to the position described above, and is asked to perform the functional test again. Both the subject and practitioner should observe any changes in range of motion and strength. If there is acute pain and inflammation in the area of the corocoid process, then the process needs to be repeated several times to release the tension in the area through incremental improvement. Practitioner should instruct subject to ice the area following treatment. This manipulation can be extremely painful for subjects who have been injured or had this problem for a prolonged period, so it is important to respect the subject's limitations with respect to treatment.

With subject still prone on table 130, practitioner will use contralateral hand 288 to gently dislocate humerus 286 while cupping the ipsilateral hand 290 around the coracobrachialis 206 muscle and deflecting that tissue in a posterior rotation 300 (as seen in FIG. 28).

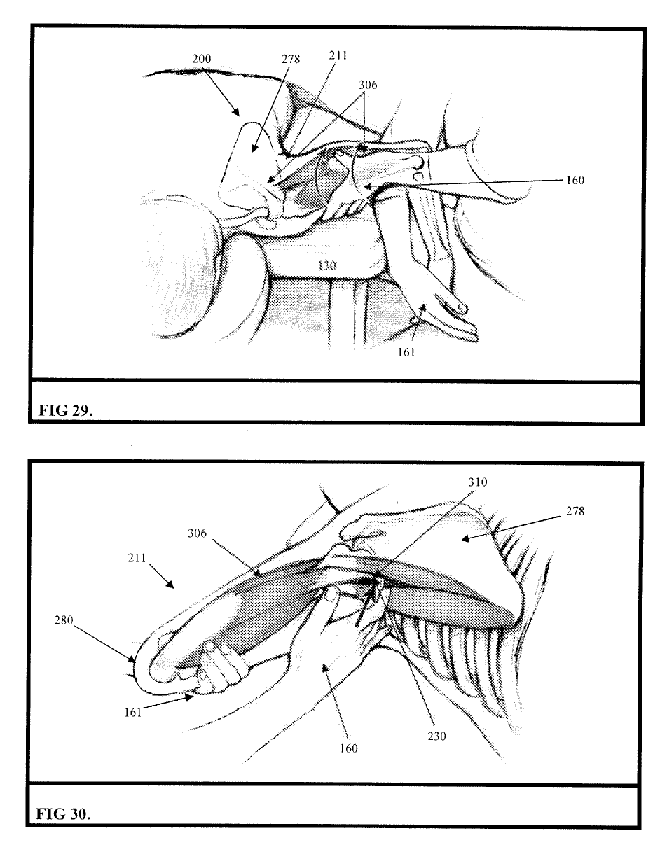

As shown in FIG. 29, for the triceps component of the treatment, the subject is still prone. The arm is positioned in full abduction, with the elbow loosely flexed to 90°. In this position, the practitioner 20 uses one hand 160 to apply perpendicular force 304 to the triceps 306 muscle, starting at the arm pit and progressing down the arm in the distal direction. The free hand 161 can support and immobilize the arm 210 at the elbow. Following this tricep work, as shown in FIG. 30, the practitioner uses firm middle finger 230 pressure to release tension in the origin of the long head 310 of the triceps brachii 306 while dislocating the upper arm 211 in the lateral direction. The origin of the long head of the triceps brachii 306 can be accessed through the space between the teres minor and major with the upper arm 211 in this position.

### Example 6

Muscles of the lower anterior abdomen: The muscles of the lower abdomen provide for stability of the trunk and pelvis, including the lower spine, and also contribute to the control of movements that cross the hip level (abduction/adduction, flexion/extension, rotation). This functional group includes the psoas major and psoas minor bilaterally. These muscles originate on the transverse processes of the lumbar vertebrae, and insert with the iliopsoas tendon at the lesser trochanter of the femur.

Functional deficits: Deficits in this group are the result of chronic and progressive lateral deflection of segments and supportive soft tissues. In all cases, the tissues in question can be tense and sensitive to the touch. Extreme lateral deviation of the psoas group is often accompanied by a flaccid nature in the muscle, with limited activation in all uses. Functionally, deficiencies in psoas activation may lead to a wide range of problems in the back and pelvis, including intervertebral disc protrusion, sciatica, sacroiliac joint pain. Psoas deficits in alignment and activation may also lead to over-recruitment of the muscles in posterior trunk and pelvis and aggravate spinal disorders, such as osteoarthritis, scoliosis, and spinal stenosis.

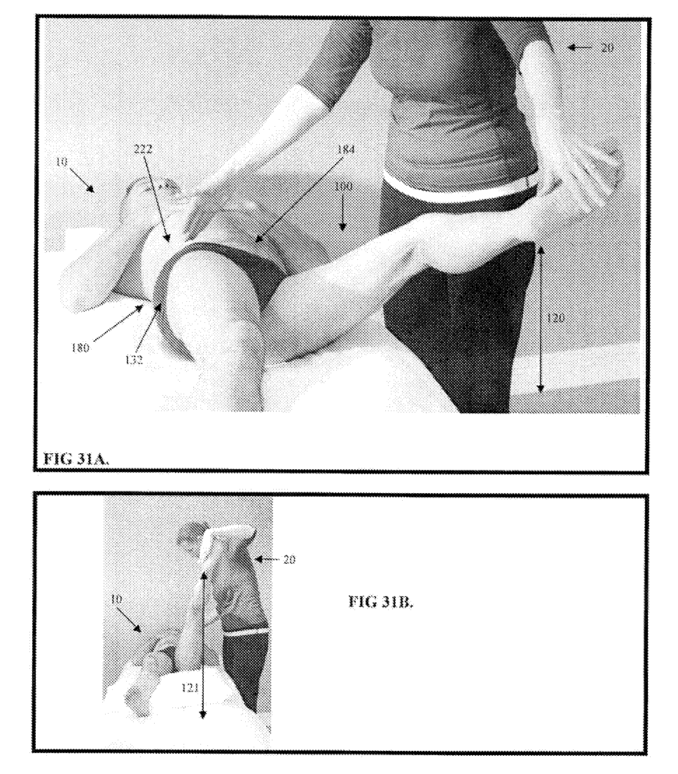

Functional assessment: Pre- and Post-intervention: The functional assessment for this group is illustrated with reference to FIGS. 31A & B. FIG. 31A shows a supine single leg raise (hip flexion and abduction). Pre-intervention, the leg raise will be of minimal amplitude 120 in both flexion and lateral directions, with inconsistent control and limited resistance to fingertip pressure applied to the foot 242 or shank 143 by the practitioner 20. The leg may feel heavy to the subject 10. In addition, compensation can be observed at the pelvis 184 and pelvic girdle 182. Specifically, trunk 222 will become over-involved in raising and balancing the limb 100, such that diminished lumbar lordosis and excessive pressure between the table 130 and the pelvis 184 on the raised leg side. The subject should make mental note of these challenges.

As shown in FIG. 31B, this resistance should improve in magnitude and consistency following intervention, and the amplitudes of hip 132 flexion and abduction should increase. Resistance to finger tip pressure should also be greater. This test should be experienced as a solid activation of the core muscles. In parallel, less movement in the trunk should be observed, with the lumbar region maintaining its moderate lordosis and both sides of the pelvis 184 remaining in level contact with the table 130. To the subject 10, the leg 100 will feel lighter and easier to control. The subject 10 should make mental note of this new posture and control, with focus on the activation of psoas 207 musculature that is a primary contributor to this action.

An integral part of intervention is educating the subject to notice the difference in muscle activation during external rotation, as compared to internal rotation. Reinforcing these activation patterns will allow subjects to develop healthy patterns of movement for the thigh 152 and lower leg or shank 142. It is advised to instruct the subject 10 to perform the following leg lift routine daily, repeating the movement 10 times for the first 3-6 weeks, and then occasionally thereafter if low back pain returns. Ask the subject to palpate the abdomen with their fingers to feel the psoas 207 engage while lifting the leg (hip flexion and abduction) without rotating the femur laterally. The subject is asked to note the ease with which they can perform this activity in contrast to the pre-intervention functional test.

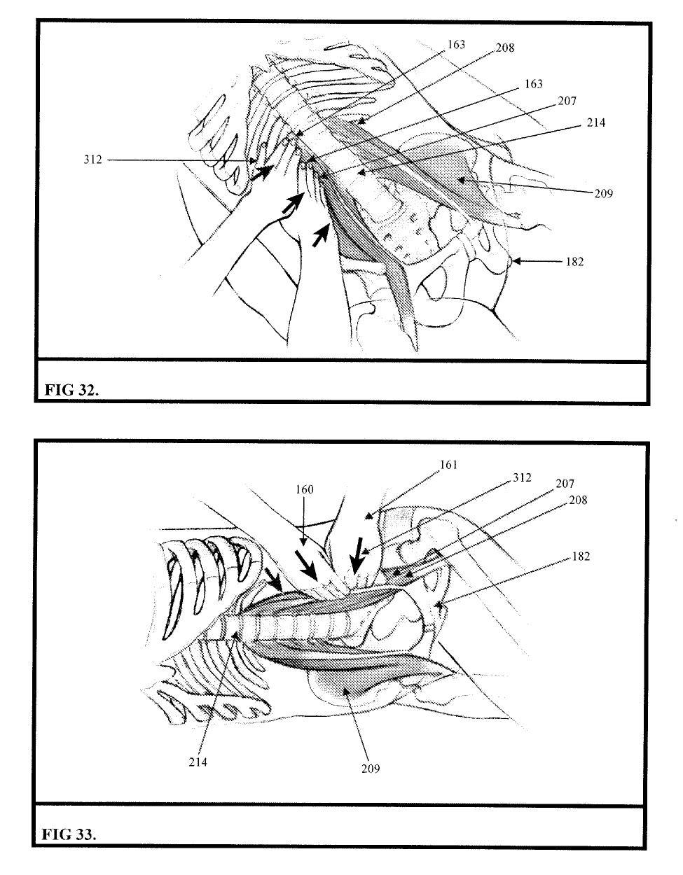

Intervention: As illustrated with reference to FIGS. 32-35, with subject supine on the table, practitioner 20 is positioned ipsilateral to the side being worked on. Due to the deep position of the psoas 207 musculature, it is important for the subject 10 to be relaxed during this treatment, so that guarding in the superficial muscle layers is minimized. The practitioner will work with the finger tips 163 of both hands together, applying pressure 312 through the abdomen to the lateral edge of the psoas major 208. Once the lateral line of the psoas 207 is identified, the practitioner applies force 312 perpendicular to the length of the psoas 207, adjusting it back toward the mid-line 260. This perpendicular force application starts at the T12 level of the psoas 207 and proceeds stepwise down to the lowest levels of the psoas 207 (as seen in FIG. 32). Multiple passes may be required on each side to provide functional improvement during the initial subject treatment, though fewer passes may be required in subsequent visits. This manipulation can also be performed with subject on their side (as shown in FIG. 33), increasing practitioner's access to the psoas 207 and allowing the practitioner to direct applied force in a more powerful downward direction.

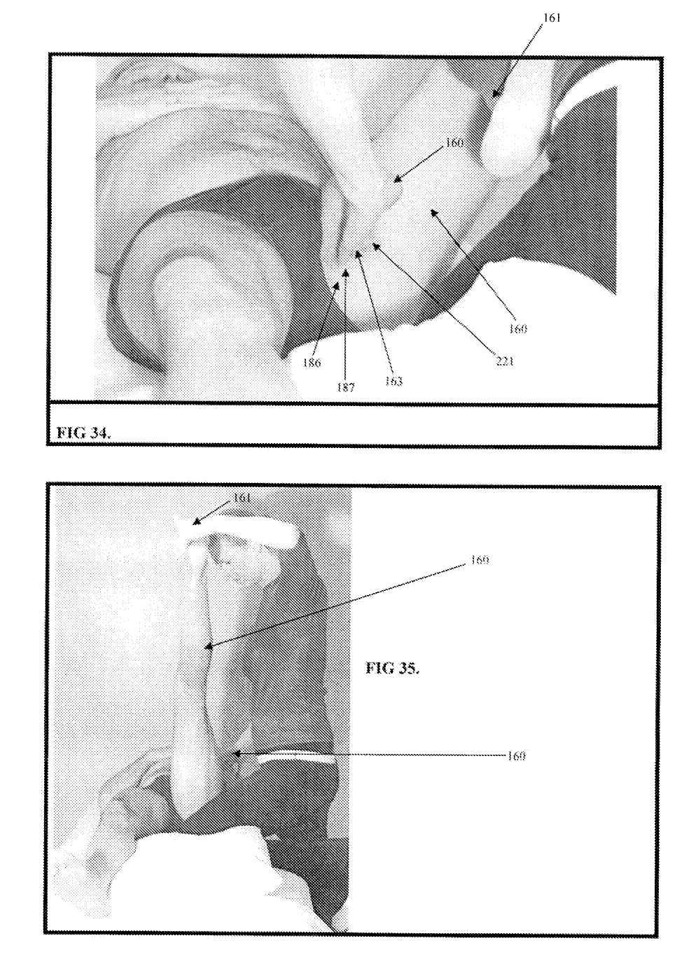

For the second component of the treatment, illustrated with particular reference to FIG. 34, subject 10 is still supine, with one hip flexed and knee bent. The practitioner uses their free hand 161 to apply pressure to the top of the subject's elevated shank. In this position, the practitioner can use a single hand 160, with the middle three fingers combined into a single-point pressure source, to locate a small gap between the upper aspects of the semimembranosus 186 and gracilis 185 in the caudal groin. Pressure through this gap reaches the lowest connection of the psoas insertion point 221, and releases tension at this point. This alignment also releases muscle tension on the head of the femur, with subjects frequently noticing greater ease of leg movement. As a second part of the upper shank alignment, explained with reference to FIG. 35, the subject extends the leg 100 to 90 degrees of hip flexion or nearest, and maintains this leg position against moderate pressure to the medial aspect of the upper leg from the practitioner. This test should be experienced by the subject as a solid activation of the core muscles. If there is a deficit in this activation, the alignment is repeated until desired results are achieved. This treatment also makes it easier for the practitioner to work above the pelvic bone line, again through decreased axial tension. The pressure from the practitioners free hand 161 helps to increase access to this connection point by stretching and flattening the semimembranosus 186 and gracilis 185. This intervention should conclude with a final manipulation that concentrates on the psoas 207 closest to the ilium 209, where there should be at least a good finger thickness space between the ilium 209 and the psoas 207 Again, this manipulation needs to be performed on both sides of the body

### Example 7

Soft tissues of the upper lateral abdomen: The muscles of this group are serratus anterior and latissimus dorsi, which are innervated by nerves from the cervical spine (C5-C8). This region also includes tissues of the trunk frequently tasked with supporting a vertical posture, and resisting the loads acting on the trunk due to the force of gravity. Sustained poor postures and asymmetrical positioning can lead to unilateral or bilateral approximation of the ribs. This decreased vertical space can cause subsequent impingement and activation problems in both the serratus and the latissimus dorsi, leading to reduced arm strength and increased loading of the shoulders.

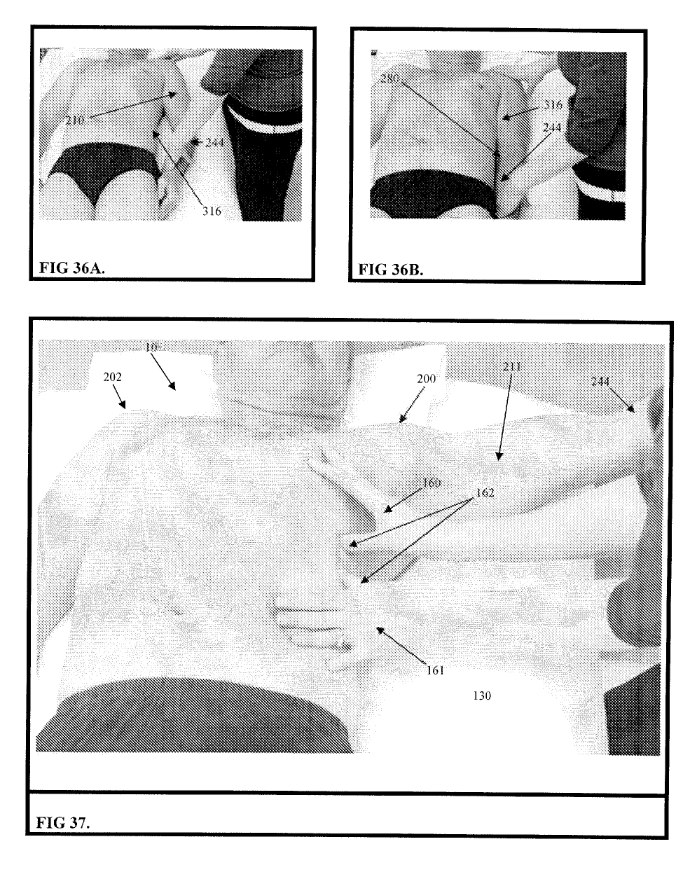

Functional assessment: Pre- and Post-intervention: The functional assessment for this group is a supine arm posture, generally illustrated with reference to FIGS. 36A and B, wherein the arm with fully extended elbow is held in the neutral position alongside the trunk. Pre-intervention, as shown in FIG. 36A, the arm 210 in this position will present limited resistance to an abducting force at the forearm 244 provided by the practitioner. During relaxed observation in this posture, an oversized gap 316 will be observed between the trunk 222 and the arm 210, and the elbow 280 and possibly wrist 282 will not be in close proximity to the trunk.

As seen in FIG. 36B, this gap 316 should decrease in magnitude following intervention, and the resistance to finger tip pressure in the abduction direction should also be greater. The subject 10 should make note of these changes.

Intervention: With subject 10 supine on the table 130 and upper arm 211 fully abducted and the forearm 244 above the head 318, practitioner uses thumb-tip 162 pressure at the midline to expand gap between ribs. Note: often there is extreme sensitivity in the location of rib compression so caution the subject that the alignment may be sensitive, and work with long slow breathing while the alignment is being done. It will be easily noted by the subject that upon the follow-up session the rib pain has diminished. Starting at armpit 320 and progressing toward the floating ribs, the practitioner can also provide gentle pressure along the rib gap towards the posterior attachment.

### Example 8

Soft tissues of the shoulder girdle: The muscles of this group are the pectoralis major, the pectoralis minor, and the deltoid, along with the latissumus dorsi on the posterior side. Soft tissues include the connective tissues for these muscles, along with the coracohumeral ligament and the ligaments of the articular capsule.

Functional deficits and pathologies: Deficits in this group are the result of chronic progressive medial deflection of the shoulder, caused by repetitive and/or sustained use in anterior work zones. Chronic loading will often lead to cumulative deficits in strength, as well as pain and discomfort. Acute injury can also lead to deficits in strength and range of motion at the shoulder.

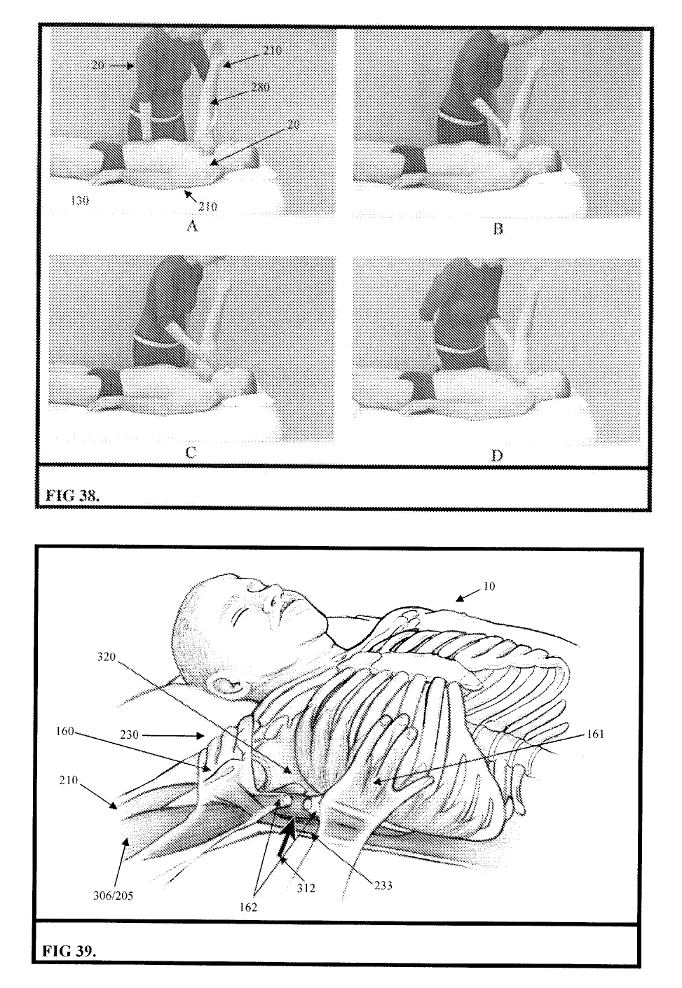

Functional assessment: Pre- and Post-intervention: The functional assessment for this group is illustrated with general reference to FIGS. 38 A to D, requires the subject to be supine, with elbow 280 fully extended and shoulder 200 flexed to 90 degrees. In this position, the practitioner 20 can apply light pressure to the subject's arm 210, and examine their resistive force produced separately in the shoulder flexion (as illustrated in FIG. 38A), extension (as illustrated in FIG. 38B), abduction (as illustrated in FIG. 38C), and adduction (as illustrated in FIG. 38D) directions. Subjects should also make note of their force production and stability of force production in each of these directions. A deficit in the alignment and control of this group may be evidenced as irregular control of the arm 216, with greater effort required to stabilize it against directive force from the practitioner. Over-contribution from the trunk 222, including axial rotation at the shoulders 200,202 and hips 132, will also be observed.

Following the intervention, the subject's resistance in all four movement directions to finger tip pressure should be greater. Contributions from the trunk, including axial rotation and grinding of the posterior shoulder and hip into the table should also be reduced. The subject should make note of these changes.

Intervention: As explained with reference to FIG. 39, with subject 10 supine on the table 130, shoulder 200 flexed to 180 degrees and elbow 280 fully extended, the practitioner can re-align the latissimus dorsi 233 and pectoralis minor 235 by applying fingertip 230, 162 pressure through the armpit 320 at the level of the third and fourth ribs. This re-alignment deflects the latissimus in a medial direction, helping to increase the postero-lateral deflection of the shoulder.

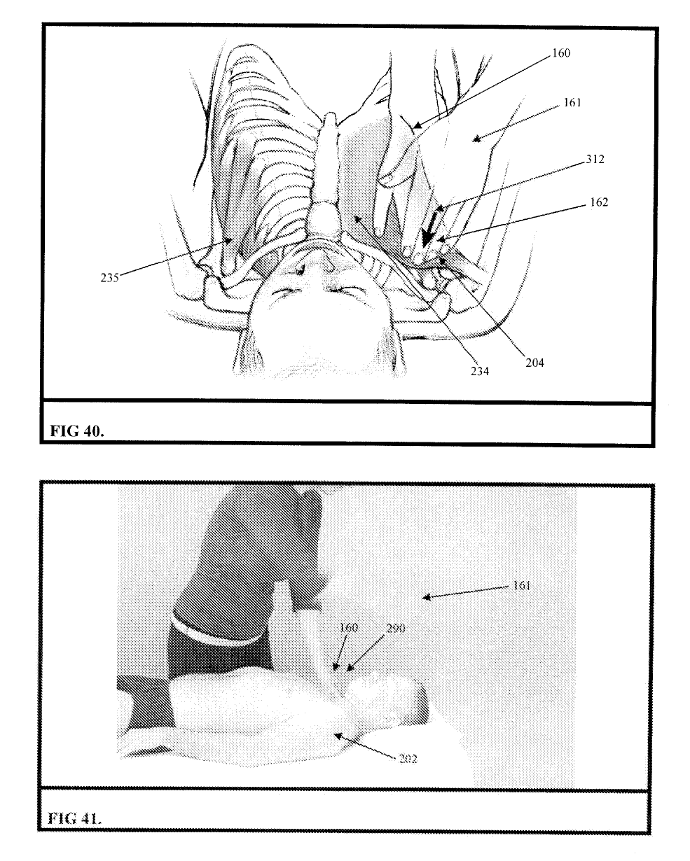

In the second component of this intervention, illustrated with reference to FIG. 40, the supine subject 10 will return their straight arm 210 to a position along the side of their body (shoulder flexed to 0 degrees). In this position, the practitioner will use both hands 160, 161 as a single-point source, applied to the insertion of the pectoralis major 204 and deflecting it in the cranial direction. This alignment releases tension in the pectoralis, and allows the shoulder complex to return to a more postero-lateral neutral position.

The practitioner will also provide a further manipulation to the shoulder 200 with the subject in the supine position with arm at the side. Both of these adjustments help to release and realign the supportive tissues in the articular capsule, and return the shoulder 200 to a more neutral position. In the first adjustment, shown in FIG. 41, practitioner 20 will cup their ipsilateral 290 hand on the subject's shoulder 200, then bring their free hand 161 up then down on the cupped hand in a swift blow. Practitioner needs to maintain a fully extended elbow in the striking arm, using the full arm as a pendulum to strike the cupped hand.

### Example 9

Soft tissues of the forearm: The tissues of the forearm provide for the large range of coordinated movements available at the wrist and in the fingers. This functional group includes the flexor and extensor muscles of the forearm, as well as the pronator teres and palmaris longus. The extensor muscles are innervated by the radial nerve and the flexor and anterior compartment muscles are innervated by the median or ulnar nerves.

Functional deficits and pathologies: Deficits in this group are the result of chronic tensile loading on soft tissues of the forearm, caused by repetitive and/or forceful use in awkward posture, or by an acute injury to the tissue(s). Chronic loading will often lead to cumulative deficits in strength, as well as pain and discomfort. These painful sensations can be constant or intermittent, given the impingement of peripheral nerves that can occur at the wrist. Often the subject will experience the forearm pain when trying to sleep.

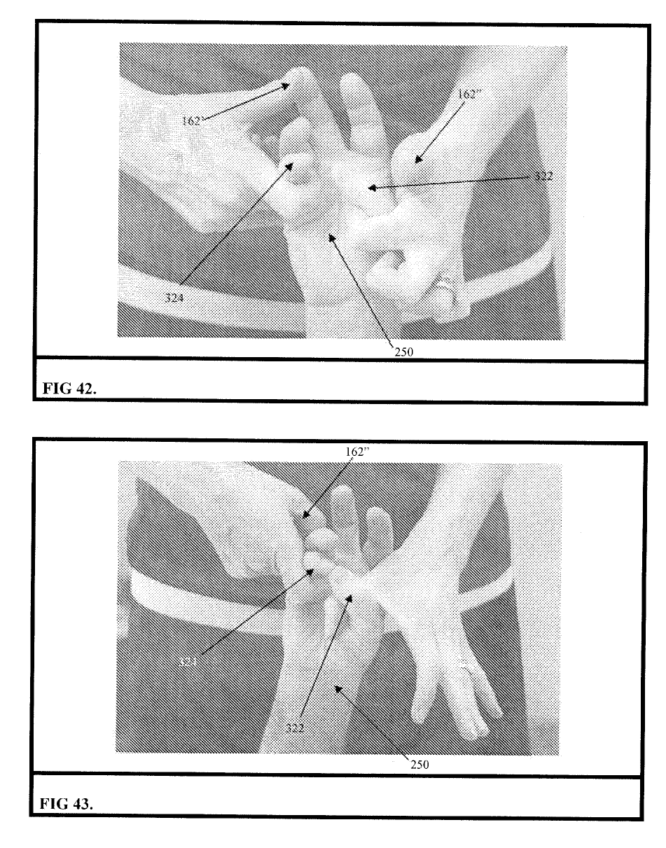

Functional assessment: Pre- and Post-intervention: The functional assessment for this group is illustrated with reference to FIG. 42 is a static contraction of the opponens pollicis muscle, drawing the thumb 322 into opposition with the smallest digit 324. This should be done with the subject 10 lying supine, upper arm 211 relaxed at side and elbow 280 flexed to 90 degrees. Pre-intervention, the subject may have difficulty bringing these digits together in opposition. Most commonly, they will be unable to keep the digits in contact when the practitioner provides a light lateral distractive force on each digit.

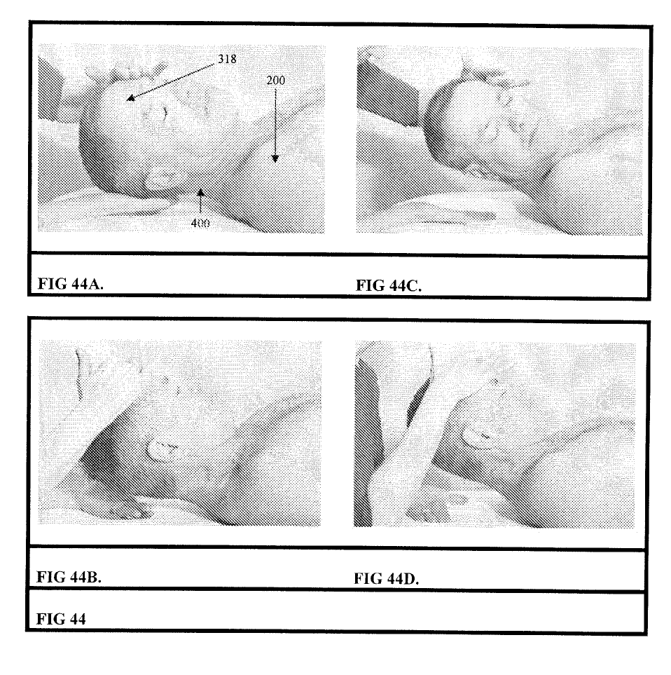

Following the intervention, as shown in FIG. 43, both the difficulty in opposing the digits and the limited resistance to distracting force should improve. Subjects should also experience a noticeable decrease in pain and an increase in grip strength.

Intervention: With subject 10 sitting on the edge of the table 130 (or supine if preferred), practitioner 20 is positioned ipsilateral to the side being worked on. Practitioner will place both hands on the subject's arm, which is slightly flexed at the elbow. Practitioner will have their working hand (hand of same side as that being worked on) on the underside of the subject's upper arm, proximal to the elbow. The practitioner's free hand will be on the lower arm, at the forearm bulk of the flexor/extensor musculature. In this position, practitioner will provide a lateral force to the upper arm, a medial force to the forearm, and simultaneously extend the subject's elbow 280 to full extension in a forceful and safe fashion. This manipulation helps to decompress and re-align any impingement at the elbow.

In the second component of the treatment, subject is still seated on table edge, facing practitioner, with arm slightly flexed at elbow 280 and forearm 244 pronated. Practitioner places both hands 160, 161 under dorsal surface of subjects hand 250, with the thumbs wrapped around to press the palmar surface of the wrist 282. In this position, practitioner extends wrist 282 while simultaneously distracting the wrist joint and pressing both thumbs into the palmar wrist surface in both the perpendicular and proximal directions. This manipulation serves to decompress and align the soft tissues at the wrist. Complete the intervention with alignment of the shoulder and neck following the instructions of those sections.

If there is still some pain or tension in the forearm, an additional alignment process should be followed. With the forearm 213 of the patient pronated, the practitioner uses a thumb to apply perpendicular pressure along the lateral edge of the supinator, moving along the length of the muscle and applying force in the medial direction. This can be painful during application, so the subject should use breath work in parallel with the adjustment. Perform a post-manipulation assessment so subject can note increases in grip strength and reduction of pain.

### Example 10

Muscles of the neck: The muscles of the neck include the three aspects of the scalene (anterior, medius, and posterior), the sternocleidomastoid and the splenius capitis. These muscles flex the head in the anterior and lateral directions, as well as providing for rotation of the head through the neck. Innervation for the muscles is through the cervical spinal nerves.

Functional deficits: Functional deficits in this group may be the result of repetitive, prolonged, and often asymmetrical deflections of the neck. Chronic deficits can also result from an acute injury. Assymetry can lead to an imbalance between bilateral muscle partners. This asymmetry may be noticed as a difference in the relative hardness of a bilateral pair of muscles, or limitations in flexion or rotation of the neck in either anterior or lateral directions. A further functional concern may be pain referred from the medial scalene into the upper parts of the biceps or coracobrachialis causing shoulder complications. Pain can also be referred cranially, particularly to the temporomandibular joint, which can subsequently lead to chronic headaches and inner ear disruptions.

Functional assessment: Pre- and Post-Intervention: There are two functional assessments for the soft tissues of the neck 400, both performed with the subject in the supine position. In the first assessment, described with particular reference to FIG. 44A, the subject rotates their head 318 around the transverse axis alternately in both directions (towards both shoulders 200, 202). Subject and practitioner should both notice the degree of rotation achieved, relative to the shoulder 200. In addition, the subject should be noticing the muscle activations that produce these rotations. In the second assessment described with particular reference to FIG. 44B, the subject will flex their head forward while in the supine position.

Pre-intervention, subjects may demonstrate a limited range of motion for supine head rotation around the transverse axis, along with discomfort in the active soft tissues near the end range of motion. For the supine neck flexion, subjects may have limited control of the head's position, evidenced through general weakness in sustaining head position, inconsistent control when maintaining a static neck posture, and low resistance to a gentle applied force from the practitioner on the forehead. In addition, the subject's face may remain in a relatively horizontal orientation during the movement, and the amplitude of the flexion may be small. Post-intervention, transverse rotations of the head may allow a greater range of motion, without discomfort near the end range, as illustrated in FIG. 44C. For the neck flexion assessment, the subject may achieve a full curled position of the neck, along with their chin tucked closer to their chest as illustrated in FIG. 44D. Finally, the subject may have improved control of static neck postures, and increased resistance to gentle deflective force from the practitioner.

Intervention: With subject supine on the table, practitioner is in a seated position at the subject's head.

### a) Alignment for Sternocleidomastoid and Infrahyoid Muscles:

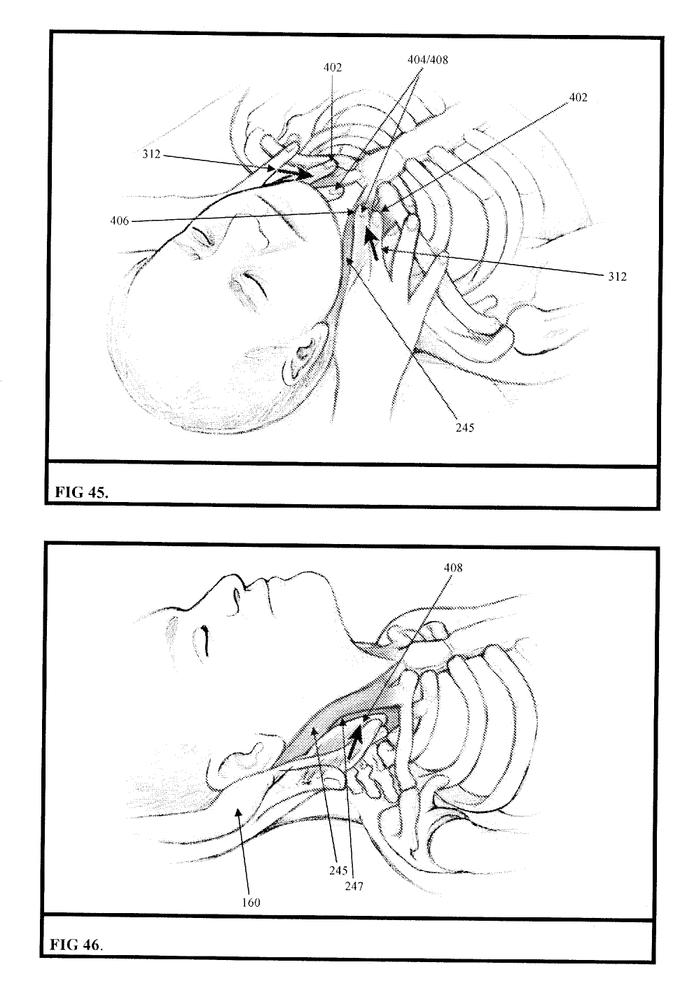

The practitioner will start by applying pressure 312 with the middle and index fingers 402 and 404 in the space 406 between the first and second rib, just lateral to the sternum. Pressure should be applied in a medio-posterior direction, to help activate the sternocleidomastoid 245. The practitioner 20 will then move their fingers to the superior surface of the clavicle, and press in a posterior direction to relax sternocleidomastoid 245 and infrahyoid 246 muscles as illustrated in FIG. 45. Practitioner will then walk fingers up the front sides of the neck, applying gentle pressure 408 to continue relaxing the hyoid. With fingers just posterior to the sternocleidomastoid 245, practitioner will apply pressure in the inferior and anterior direction, working to deflect the anterior scalene 247 muscles to a more medial position as illustrated in FIG. 46. To ensure that alignment is complete to the sternocleidomastoid 245 the subject is asked to lift the head tucking the chin. The subject is observed for ease in the flexion, and strength is tested with a light pressure on the forehead. This manipulation should be repeated until substantial improvement is achieved. Both practitioner and subject should easily observe an improvement.

### b) Alignment of Scalene Muscles

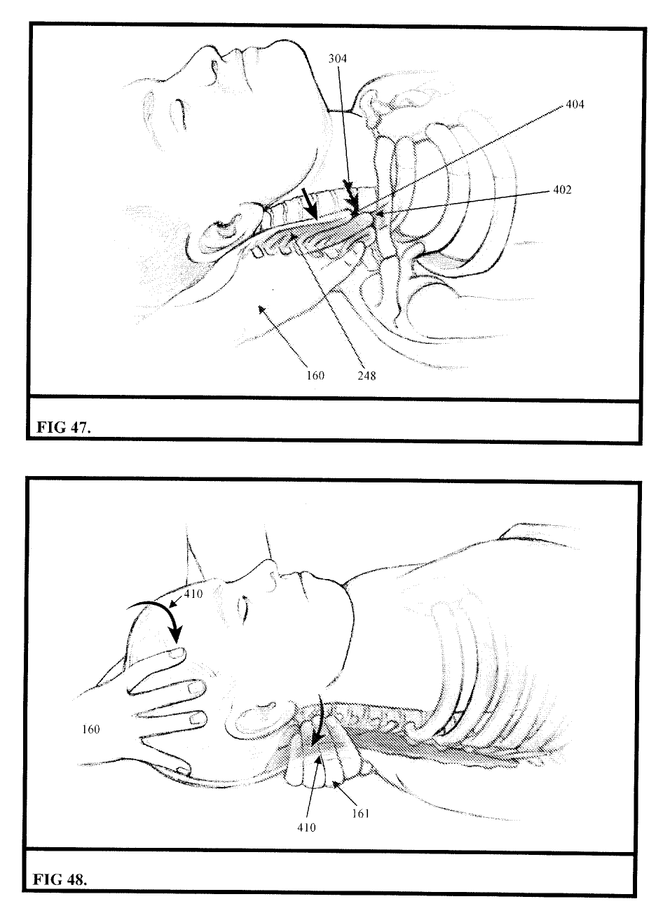

The medial scalene can be located by walking the fingers back from the anterior scalene. With two fingers 402 and 404 on the medial scalene 248 on each side of the head, practitioner 20 will push down and posteriorly on the muscle starting at the clavicle level and walking up, always keeping the fingers perpendicular to the muscle as shown in FIG. 47. Practitioner should take special note of any deflection asymmetries here, and apply differential force as appropriate. This activation can be uncomfortable for subjects. It may be important to warn subject in advance that they may experience discomfort that extends into the scalene's insertion at the 1^{st} and 2^{nd} rib and into the upper shoulder and arms. To ensure that the muscles have moved into position the practitioner may ask the subject to repeat a rotation of the head in each direction to observe any changes in degree of rotation. If more correction is required the steps are repeated until satisfactory results are achieved. The subject should note decreased discomfort and fewer limitations with each repeat of the manipulation.

### c) Alignment of the Capitis Muscles

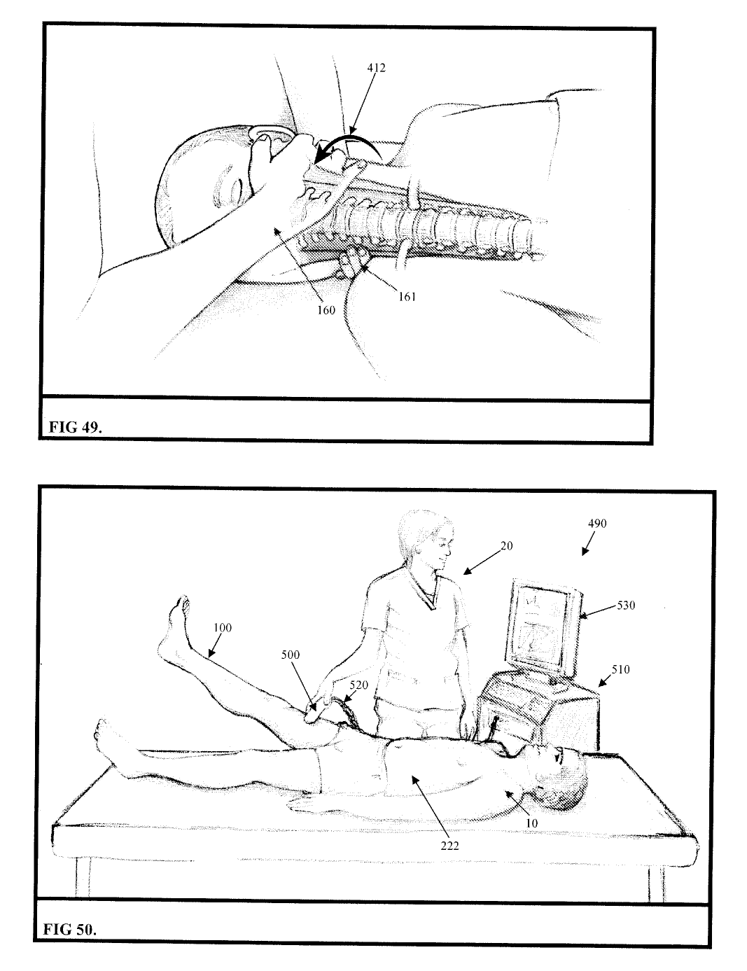

Following the deflection of the scalene, as shown in FIG. 48, the practitioner may reach under the neck 400 and curl their fingers around the splenius capitis 263 on the contralateral side. Gentle deflection of this muscle toward the midline, in combination with a lateral rotation 410 of the head toward the muscle being activated, can help deflect the splenius to a more neutral posture. As shown in FIG. 49, the practitioner 20 may leverage the jaw at the chin in the neck twist 412 of this manipulation. If there is still residual tightness along the length of the capitis 263, ask the subject to maintain a neutral head position while gently working the muscle medially along the spine from the proximal to distal end. Often the problem area is easily palpated, and the subject should communicate with practitioner to direct the process. Second functional assessment should be repeated to ensure results have been achieved.

The final therapy for this section is a long diagonal stretch for the splenius capitis 263. The practitioner is in a standing position behind the subject at the head. The subject is passive while the practitioner lifts and rotates the head laterally and in forward flexion. At the same time, the practitioner supports the head with one hand cupped at the occiput, and places the free hand on the anterior side of the shoulder opposite to the direction of rotation, pressing posteriorly while gently stretching the neck/head on the diagonal. This stretch needs to be performed in both directions. The subject is asked if there is any discomfort while in the stretch. If there is anything other than a pleasant stretch, it will be felt along the length of the capitis muscle from the occiput to the upper ribs where it attaches, and it indicates more alignment is required for the capitis muscles by moving them medially. Commonly, there is a twinge beneath the rhomboid indicating that the lower end of the capitis needs to be moved medially. This is deep work so it is important to position the fingers perpendicular to the muscle with deep pressure as the muscle is moved medially then repeat the stretch until it is pleasant and easy for the subject. Again this would be performed in both directions.

Re-education exercises: The practitioner is seated behind the subject in the first and second exercises. In the following three exercises there is a component involving breath work. The subject inhales prior to the activation and upon activation exhales to enhance the release.

In the first exercise, the subject will perform an axial rotation of the neck and head against gentle resistance from the practitioner. As the subject returns their neck/head in the direction of the opposite shoulder, the practitioner offers resistance with hand resting on the side of the face. The subject then becomes passive, and the practitioner will gently move subject's head incrementally toward the direction of the shoulder. This is generally done in three increments until full rotation is achieved to the subject's working edge of comfort and range. Note: There should be no pain in this activation. If the subject experiences pain, more alignment or further investigation of possible cervical issues may be required. This is repeated to the opposite direction. It is noted by both subject and practitioner that the range of motion has increased.

In the second exercise, the subject should think about grounding their neck and shoulders in a rigid fashion into the bed during the activity. The subject should be focusing on using muscles at the trunk's core, and specifically the splenius capitis to maintain this posture. Working one side at a time, and coinciding with an exhalation, the practitioner will provide resistance to the subject with a hand placed behind the ear on the side of the head. Note: Again, there should be no pain upon activations. If there is pain, communicate with the subject about what the nature of the pain and determine if more repositioning is required, or if further investigation of cervical issues is warranted. Upon completing activations on both sides of the neck, the subject is asked to adopt a passive control of the neck while the practitioner moves the head and neck gently from side to side. The subject should note the ease with which the head moves.

The third exercise is done with the subject maintaining passive neck control, and the practitioner lifting the subject's neck and head in gentle forward flexion. The practitioner is standing behind the subject, and the subject's head is supported with both of the practitioner's hands, placed at the base of the occiput. The subject is asked to inhale, then exhale and activate against the resistance of the practitioner's supportive hands. At the end of the exhalation, the subject is instructed to return to a passive neck control. The practitioner incrementally lifts the head and neck into increased forward flexion (to the point of pleasant tension), and the activation is repeated. At completion, rest the subject's head on the table and note the physical and functional changes in the subject.

### Sample Case Histories

Case History #1, male, age 53—Prolapsed Cervical Discs: The patient was experiencing pain at the C4-5, 6 & 7 levels. He works as a commercial heating/air conditioning contractor and has for the past 25 years worn a hardhat. When first assessed for treatment he was incapable of head extension and could only minimally rotate in either direction without extreme discomfort. After the first treatment session a significant improvement was noted in the patient's in range of motion. By the second session the patient was pain free, and the third session further increased his range of movement. The patient retains between 80% and 90% his alignments from each treatment and has received altogether four 90 minute sessions and nine 30 minute sessions of treatment. The neck problems he continues to experience are mostly due to the weight of his hardhat. He presents mild muscular neck tension in his scalenes and sternocleidomastoid which is easily relieved with treatment.

Case History #2, male, age 52—Disc Protrusion: The patient had a narrowing of the L4 and L5 disc with a slight forward shift of L4 on L5 by 5 mm. There was evidence of sclerotic facets from L4 to sacrum. When first assessed, the patient presented with extreme muscular atrophy of his left leg and buttock with a noticeable drop in his left foot. Following his first treatment, he was still in pain but the pain was reduced. After his second treatment, he was more comfortable but still in pain with light guarding. When he presented for his third session, the patient was considerably improved with only low levels of pain. Upon his sixth session, the patient was completely pain free and no longer displayed a drop in his left foot with a remarkable gain in strength.

Case History #3, male, age 72—Stenosis Spurring and Disc Protrusion: The patient has a narrowing at the L5-S1 disc space with vacuum phenomenon, sclerosis and spurring. When he presented for treatment, the patient was taking eight PERCACET a day for pain without relief. He was unable to lift his left heel when walking and unable to engage his femoral bicep during functional testing. He also presented a chronic shoulder problem. By the second treatment, the patient had decreased his pain medication to five PERCACET a day, showed improved ability to respond and was in much less pain. At his third treatment, the patient displayed greater strength and amplitude to all his functional tests and improved his ability to focus on his muscle status, he decreased his medication to two to three doses a day. By the sixth treatment, he was off all of his pain medication and was moving pain free. The patient had a total of six treatment sessions and has remained relatively pain free.

Case History #6, female, age 42—Carpal Tunnel Syndrome: The patient was suffering from a posterolateral disc protrusion at C5-6 level, resulting in narrowing of the canal to the left of the midline. She was evaluated with severe Carpal Tunnel Syndrome and underwent treatment for the injury consisting of mainly physical therapy and ultimately surgery exploring the right medium nerve in 2003. She was assessed as being incapable of returning to work and went on permanent disability. After the first treatment the patient was relieved by 50% and the further treatment resulted in complete relief. The patient can now drive, shower, brush her teeth and perform all her normal tasks pain free and her grip strength is up to normal. The patient received six 90 minute sessions and two 30 minute sessions.

Case History #7, female, age 38—Disc Protrusion: MRI studies showed a mild desiccation at L5-5 and L5-S1. There is a marked narrowing of the disc space with moderate right posterolateral disc herniation not seen in the first study and it was also learned that the patient had developed a Tarlov cyst. The patient underwent conventional physiotherapy and chiropractic treatment for the disc problem with no result. Just prior to treatment a second MRI was ordered. During the period when the patient waited for her MRI she underwent treatment. Once the second MRI was performed it demonstrated a reduction in the size of the disc from receiving the treatment. The patient has received four 90 minute sessions and eight 30 minute maintenance sessions. She will continue in active care for a few more months to ensure that she is stable and then will move to long term maintenance. The patient is considerably improved with no pain with the exception of what she described as mild back ache only during her menstrual cycles.

Case History #8, male, age 71—Scoliosis, Spinal Stenosis, Disc Protrusion, Severe Arthritic Right Hip, Spurring and Soft Tissue Calcification: When the patient presented for his first treatment session he was barely able to walk, his pain level was quite high and he was in a desperate state emotionally. After his treatment he has noticed improvement in his posture and his feet are no longer rotated externally with bent knees, he no longer requires his pant cuffs to be rolled, he walks without much sway, his limp is 50% improved and he has more energy. The patient is nearly pain free. His neck range of motion was also seriously restricted but he now has a full range of motion and is steadily improving. These results have occurred from seven 90 minute sessions. He is now on 30 minute maintenance sessions every ten days.

It will be appreciated that a variety of refinements and amendments to the foregoing embodiment will be readily recognized and implemented by those skilled in the art. Details of specific elements are disclosed herein with reference to alternative embodiments.

The embodiments presented herein are illustrative of the general nature of the subject matter claimed and are not limiting. It will be understood by those skilled in the art how these embodiments can be readily modified and/or adapted for various applications and in various ways without departing from the spirit and scope of the subject matter disclosed and claimed. The claims hereof are to be understood to include without limitation all alternative embodiments and equivalents of the subject matter hereof. Phrases, words and terms employed herein are illustrative and are not limiting. Where permissible by law, all references cited herein are incorporated by reference in their entirety. It will be appreciated that any aspects of the different embodiments disclosed herein may be combined in a range of possible alternative embodiments, and alternative combinations of features, all of which varied combinations of features are to be understood to form a part of the subject matter claimed.

The embodiments of the invention if which an exclusive right or privilege is claimed are defined as follows:

1. A method for treating a musculoskeletal dysfunction in a body region of a human, said body region comprising a first tissue and having a first configuration,
said method comprising the steps of:
a) carrying out a first functional assessment comprising:
i) causing said human to actuate said body region;
ii) observing a predetermined feature of said actuation of said body region; and
iii) comparing said predetermined feature of said observed actuation to said predetermined feature of a desired actuation to assess said musculoskeletal dysfunction; and
b) repositioning said first tissue in a second configuration to treat said musculoskeletal dysfunction.

2. The method according to claim 1, wherein said actuation comprises holding said body region substantially static and said functional assessment further comprises observing the tissue actuation required to maintain said stasis.

3. The method according to claim 2, wherein said predetermined feature is a loading pattern of said body region.

4. The method according to claim 3, wherein said body region comprises a second tissue and said repositioning comprises repositioning of said first tissue relative to said second tissue to establish a substantially stable said second configuration.

5. The method according to claim 3, further comprising
a) conducting a second functional assessment; and
b) comparing said predetermined feature of said actuation during said first functional assessment with said predetermined feature of said actuation during said second functional assessment.

6. The method according to claim 4, wherein said first tissue has a length and said repositioning comprises applying pressure substantially perpendicular to said length.

7. The method according to claim 2 further comprising training said body region to adopt said second configuration.

8. The method according to claim 7 wherein said training comprises guiding a movement of said body region to follow a predetermined pattern while causing the human to concentrate on the quality of said guided movement.

9. The method according to claim 1 wherein said treatment has a long term efficacy.

10. A method for evaluating a musculoskeletal dysfunction in a body region of a human, said body region having a tissue configuration, said method comprising:
a) causing said human to actuate said body region;
b) observing a predetermined feature of said actuation of said body region; and
c) comparing said predetermined feature of said observed actuation to predetermined feature of a desired actuation to evaluate said musculoskeletal dysfunction.

11. A method for enhancing the performance of a body region of a human, said body region having a first configuration and a first tissue, said method comprising diagnosing the actuation of said body region prior to repositioning said first tissue, said evaluating comprising:
a) causing said human to actuate said body region;
b) observing a predetermined feature of said actuation of said body region;
c) comparing said predetermined feature of said observed actuation to a predetermined feature of a desired actuation to assess said musculoskeletal dysfunction.

12. The method according to claim 11, wherein said actuation comprises holding said body region substantially static and said functional assessment further comprises observing the tissue actuation required to maintain said stasis.

13. The method according to claim 12, wherein said predetermined feature is a loading pattern of said body region.

14. The method according to claim 13, wherein said body region comprises a second tissue and said repositioning comprises repositioning of said first tissue relative to said second tissue to establish substantially stable said second configuration.

15. The method according to claim 13, further comprising
a) conducting a second functional assessment; and
b) comparing said predetermined feature of said actuation during said first functional assessment with said predetermined feature of said actuation during said second functional assessment.

16. The method according to claim 11, wherein said first tissue has a length and said repositioning comprises applying pressure substantially perpendicular to said length.

17. The method according to claim 11 further comprising training said body region to adopt said second configuration.

18. The method according to claim 17 wherein said training comprises guiding a movement of said body region to follow a predetermined pattern while causing the human to concentrate on the quality of said guided movement.

19. The method according to claim 11 wherein said functional assessment comprises the use of an apparatus to monitor said predetermined feature of said actuation.

20. An apparatus for determining an actuation pattern of predetermined musculoskeletal tissues in a human subject having said musculoskeletal tissues.
